(19) United States
(12) Patent Application Publication
(10) Pub. No.: US 2017/0269060 A1
(43) Pub. Date: Sep. 21, 2017

(54) Assays and Compounds to Treat Kidney Diseases

(71) Applicant: Rush University Medical Center, Chicago, IL (US)

(72) Inventors: Vineet Gupta, Pinecrest, FL (US); Jochen Reiser, Chicago, IL (US)

(73) Assignee: Rush University Medical Center, Chicago, IL (US)

(21) Appl. No.: 15/310,350

(22) PCT Filed: May 8, 2015
(86) PCT No.: PCT/US15/29832

§ 371 Date: Nov. 10, 2016

Related U.S. Application Data
(60) Provisional application No. 61/992,671, filed on May 13, 2014.

(51) Int. Cl. G01N 33/50; A61K 31/55; A61K 31/52; A61K 31/352; A61K 31/4418; A61K 31/472; A61K 31/517; A61K 31/5025; A61K 31/4409; A61K 31/506; A61K 31/4745

(52) U.S. Cl. CPC G01N 33/502; G01N 33/5026; G01N 33/5023; G01N 33/5032; A61K 31/506; A61K 31/55; A61K 31/52; A61K 31/352; A61K 31/4745; A61K 31/472; A61K 31/517; A61K 31/5025; A61K 31/4409; A61K 31/4418; G01N 2500/10; G02B 21/0028

(57) ABSTRACT

Methods of treating kidney disease and protecting podocytes from injury are provided. Methods of screening agents for the treatment of kidney disease are also provided. In addition, methods of identifying structural or functional defects in a patient's podocytes and methods of identifying kidney disease causing agents in a patient's biological sample are also provided.

### RELATED APPLICATIONS

This application claims the benefit of U.S. Provisional Application No. 61/992,671 filed May 13, 2014, which is incorporated by reference herein in its entirety.

### BACKGROUND

1. Technical Field

Assays and compounds to treat kidney diseases are provided and in particular, compound screening assays and diagnostic screening assays are provided. Therapeutic compounds for the treatment of kidney diseases are also provided.

2. Background Information

Kidney-failure (End stage renal disease, ESRD) is a debilitating disease with no treatments or therapeutics. Patients suffering from ESRD go on dialysis and require a kidney transplant in order to regain kidney function. A majority of kidney diseases and ESRD originate within the glomerulus and are associated with proteinuria. ESRD imposes a significant burden on the patients and the health care system worldwide in is in urgent need for effective therapies and better treatment options.

The study of kidney disease is complex because the onset is often undetected, diseases may be acute or chronic in nature, the genetic makeup of the host leads to variable clinical syndromes, and multiple organs are often involved simultaneously. Cell cultures and animal models are necessary to study disease susceptibility, mechanisms, prognosis, and potential therapies. In renal research, the use of experimental animal models has proved invaluable. Nevertheless, animal models are often limited because they do not always fully replicate the human diseases. For example, the current mouse models of diabetic nephropathy do not typically demonstrate the features of human diseases, such as Kimmelstiel-Wilson nodules.

One of the fastest moving areas of research progress in nephrology has been the appreciation of the importance of the visceral glomerular epithelial cell, hereinafter referred to as the podocyte, in health and disease. Podocytes form the final filtration barrier for blood in the glomerulus and play a central role in glomerular diseases that ultimately result in ESRD (Mundel P. and Reiser J. Kidney Int 2010, 77: 571-80). Podocytes are terminally differentiated pericyte-like cells that reside on the outer surface of the glomerular basement membrane and give rise to long major processes that branch into structures known as foot processes (FPs). FPs of adjacent podocyte cells interdigitate and form narrow filtration slits, a structure known as the slit diaphragm (SD), which forms a molecular sieve that the body uses to retain proteins in the blood, while filtering small molecules and other agents in to the urinary space. Thus, podocyte injury is a common theme in many proteinuric kidney diseases (Mundel P. and Reiser J. Kidney Int 2010, 77: 571-80). Additionally, because of this central role of podocytes in maintaining healthy kidney function, podocytes represent a key target for the development of novel therapeutics to treat a variety of kidney diseases.

Because podocytes play a key role in the prevention of proteinuria in the healthy situation, they are important targets of injury in a variety of renal diseases and are important determinants of outcome. Improved understanding of podocyte biology has come from two main arenas: first, molecular genetics of single gene disorders which lead to rare forms of congenital nephrotic syndrome; and second, focused study of this specialized cell type in vivo and in vitro. Research has also shed light on specific proteins, RNA and cell signaling mechanisms in the podocytes that represent good targets for drug discovery efforts, diagnostics and therapeutics. For example, recent studies have shown that stabilization of the podocyte actin cyctoskeleton, both in vitro and in vivo, can significantly protect the podocytes and ameliorate proteinuria in vivo, suggesting that such agents may have a therapeutic potential for treating proteinuria in humans.

Although podocyte cultures do not fully replicate the in vivo environment, they have several major advantages. These include the ability to directly study mechanistic events, to control the environment such that specific hypotheses can be tested, and that multiple experiments can be performed to validate the initial observations.

Currently, a significant limitation in using podocyte cultures is the difficulty in handling podocytes and using them in a medium- or high-throughput assay environment (Reiser, Gupta et al, Kidney Int. (2010) 77, 662). Assays for systematic, high-throughput screening of libraries of potential agents are not available. Podocytes are a challenging cell type for use in such assays.

A need exists for developing podocyte cultures that are suitable for drug screening (such as high throughput drug screening), for studying molecular pathways involved in glomerular diseases and for assessment of patient samples.

A need also exists for therapeutics targeting podocytes to treat a variety of kidney diseases.

### BRIEF SUMMARY

Methods of treating kidney disease and protecting podocytes from injury are provided. Methods of screening agents for the treatment of kidney disease are also provided. In addition, methods of identifying structural or functional defects in a patient's podocytes and methods of identifying kidney disease causing agents in a patient's biological sample are also provided.

### BRIEF DESCRIPTION OF THE DRAWINGS

FIG. 1A-1D. PAN induces dose-dependent podocyte damage that can be quantitated in the HCS assay using a variety of parameters. Puromycin aminonucleoside (PAN) is an established podocyte toxin and is used in many in vitro and in vivo studies (22). FIG. 1 illustrates the results of differentiated murine podocytes cultured in multiwall plates and treated with an increasing dose of PAN and incubated at 37° C. for 48 hours. Cellular damage was assessed at 48 h. A-D. Dose-response curves using various parameters show a dose-dependent change and PAN-induced podocyte damage. Each data point represents mean±SE of 3-6 wells and is representative of 2-3 independent assays.

FIG. 2A-2D. Mizoribine dose-dependently prevents PAN-induced podocyte damage.

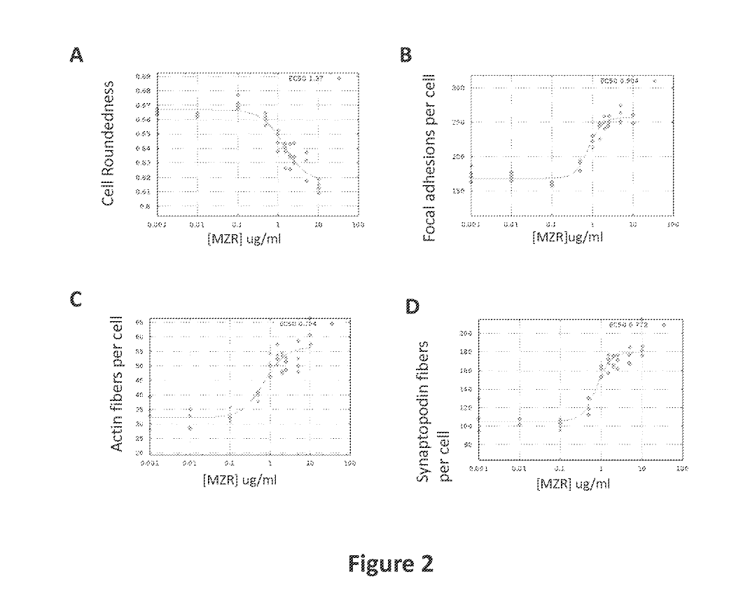

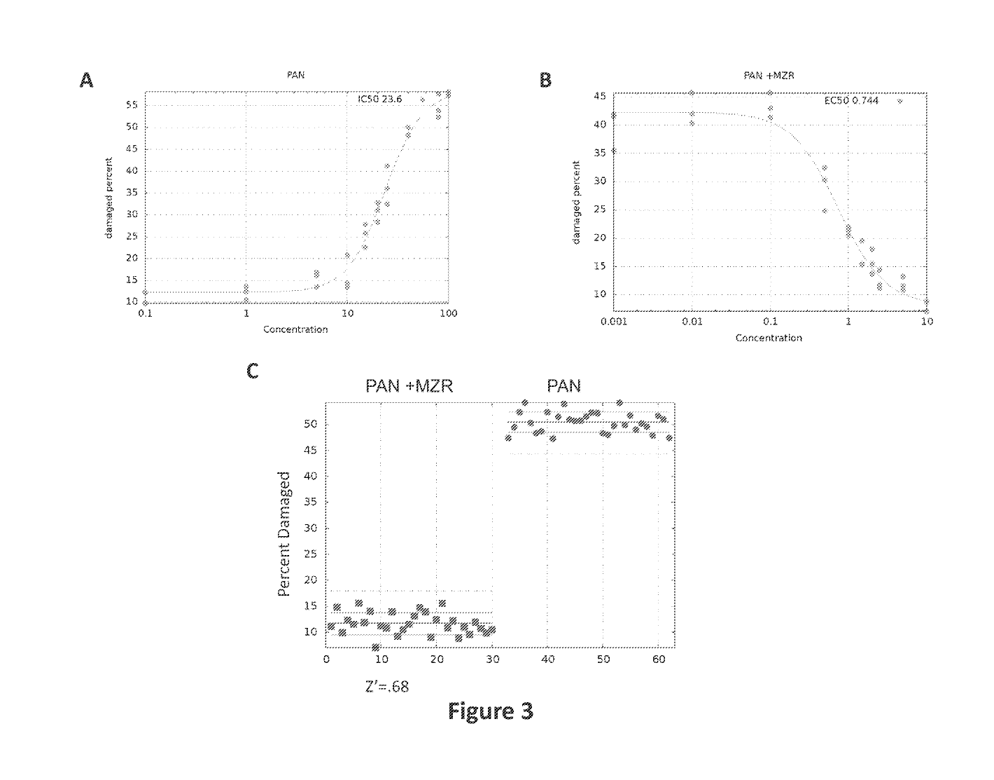

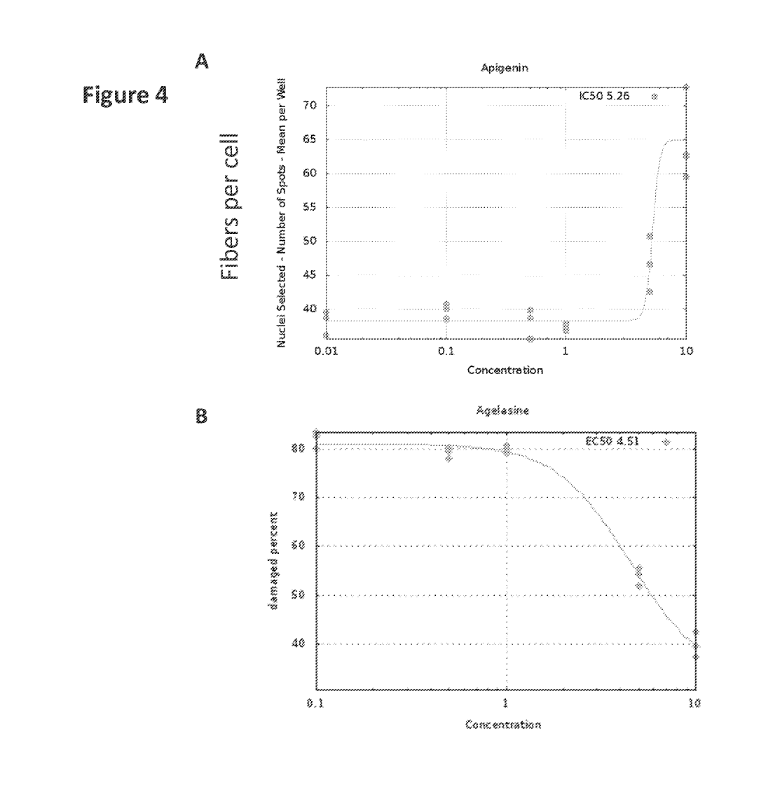

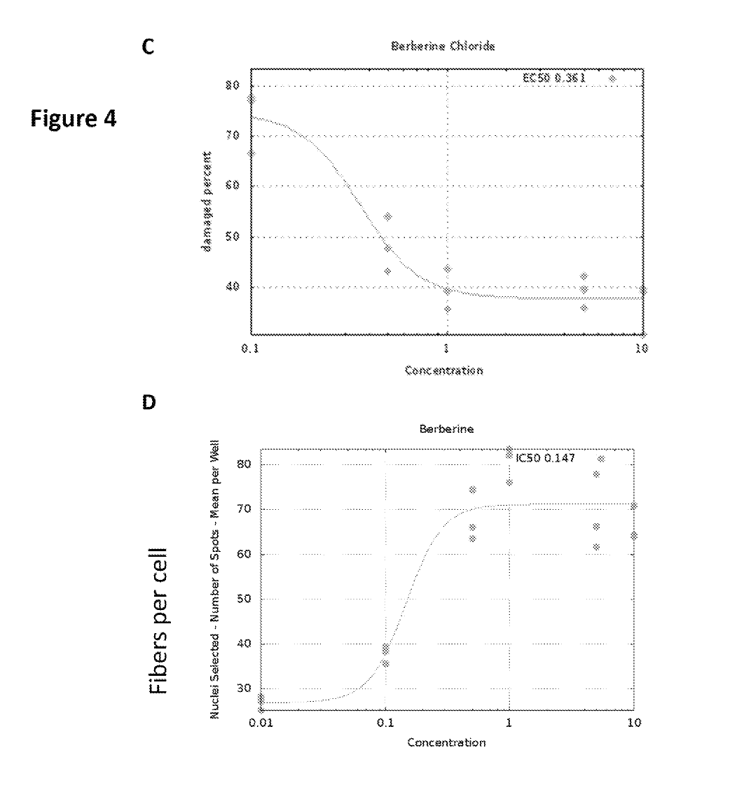

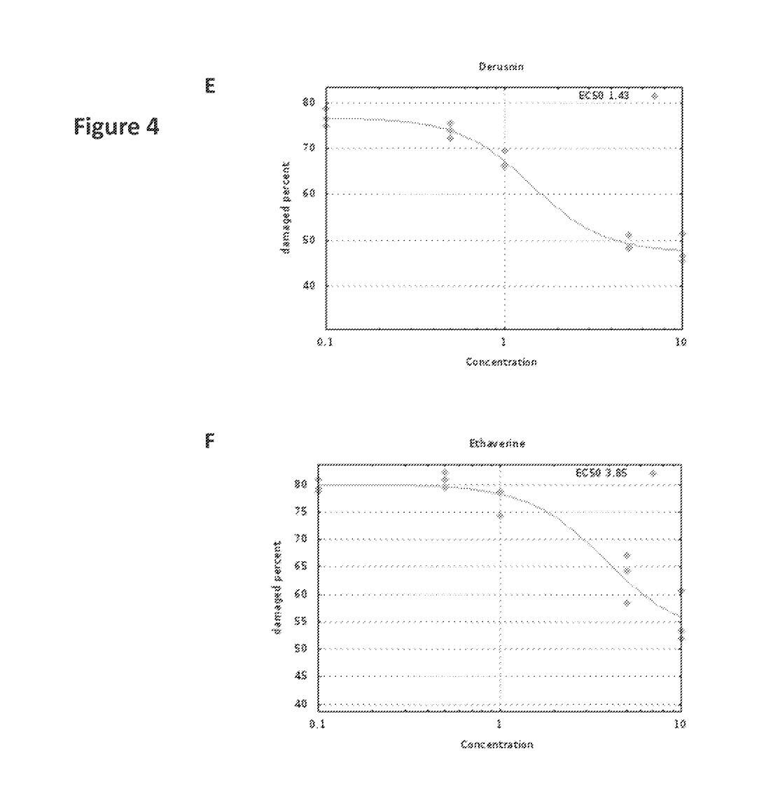

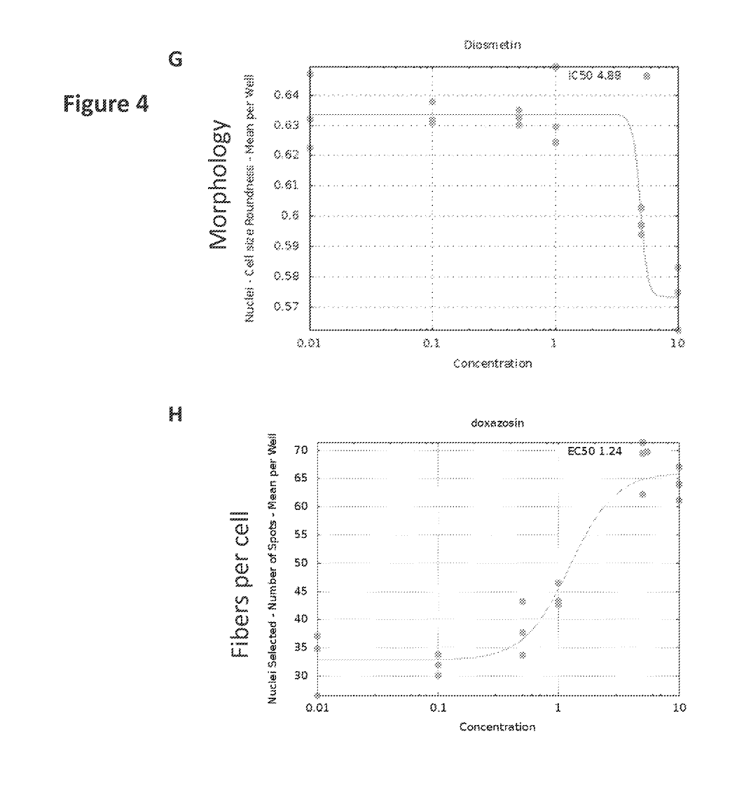

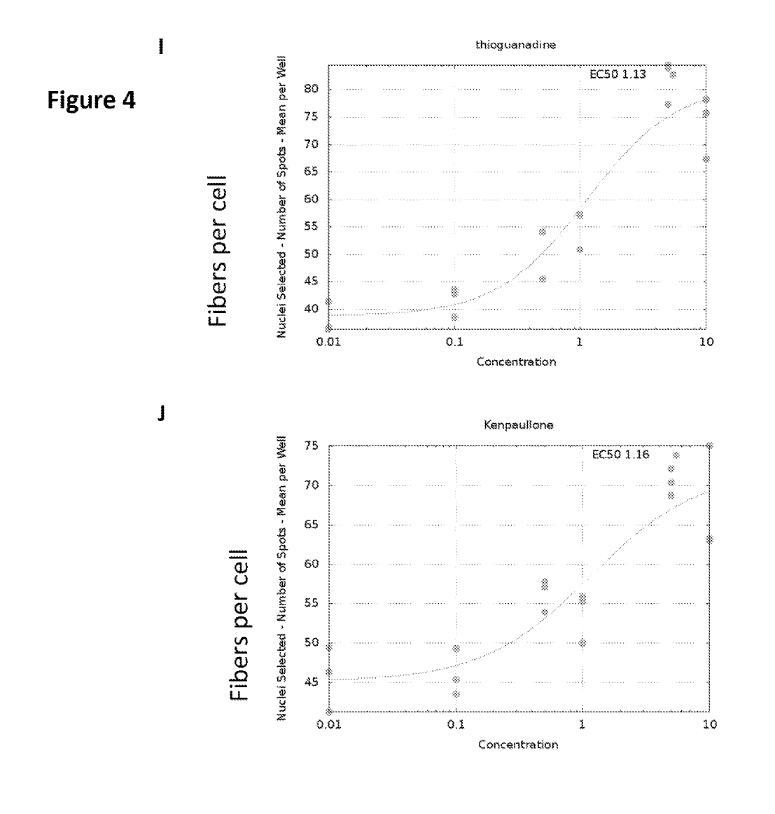

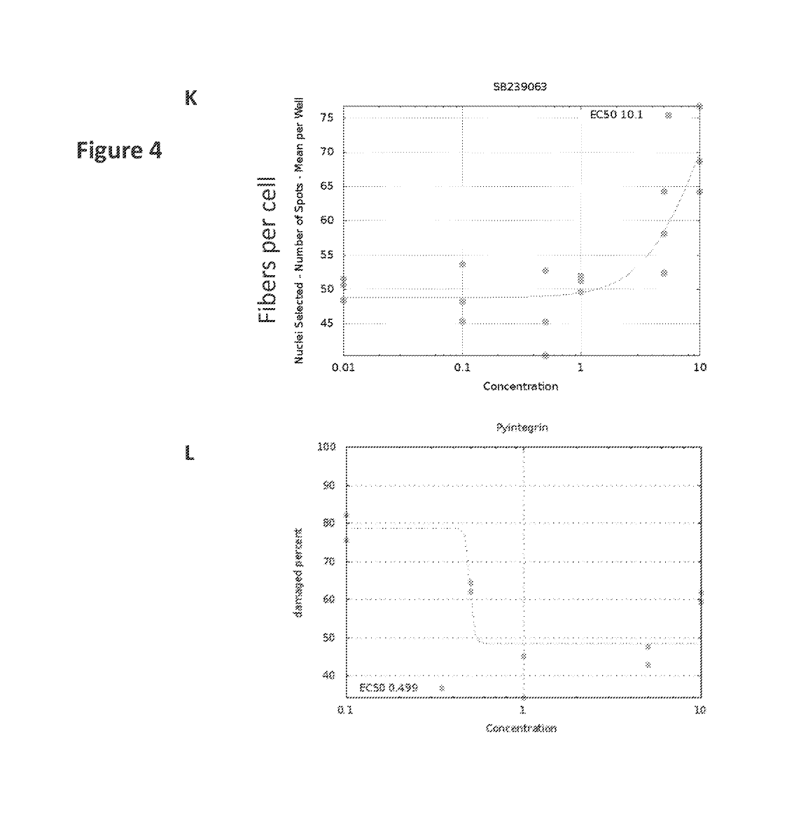

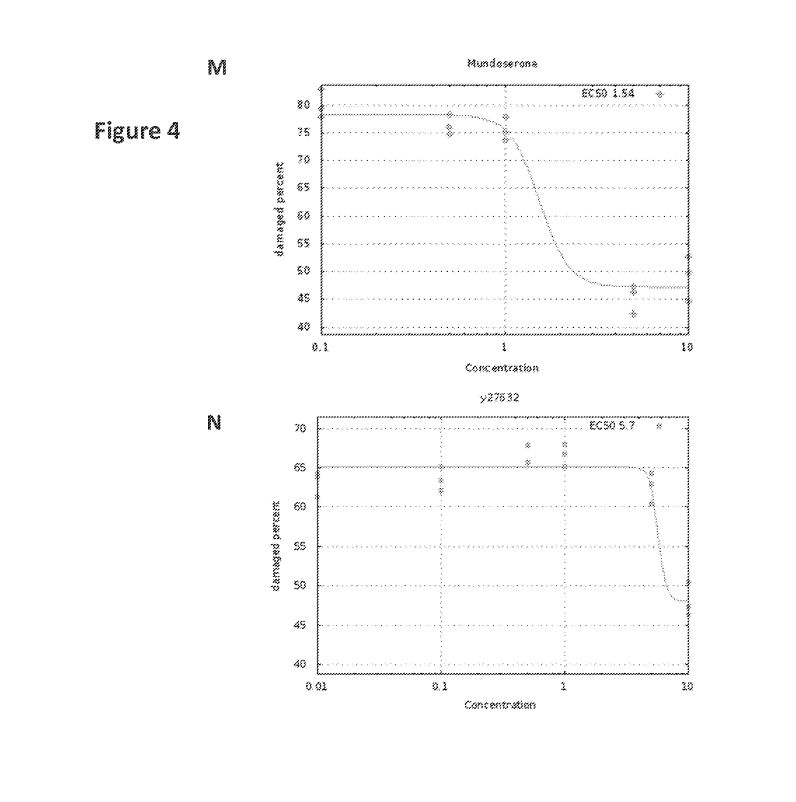

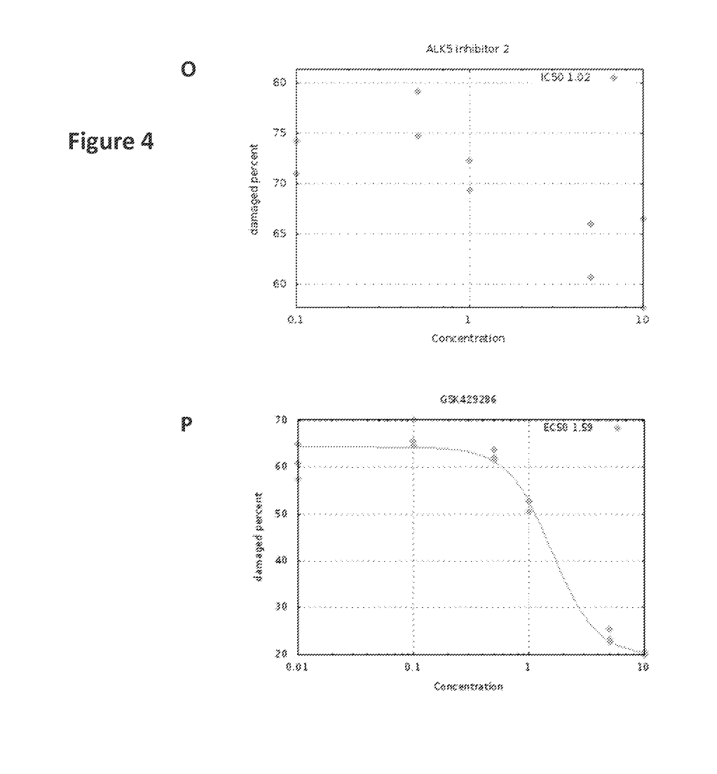

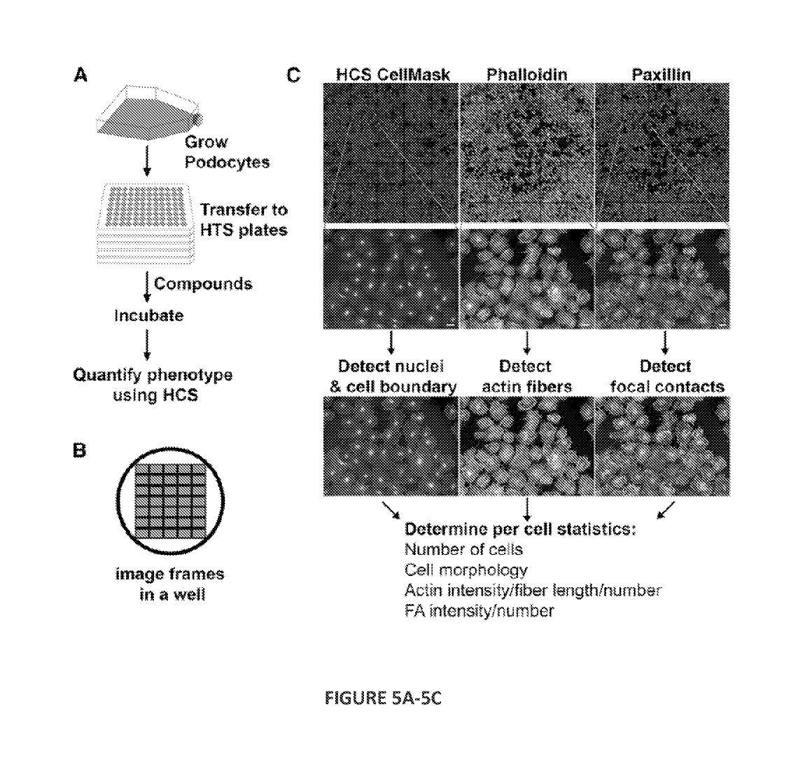

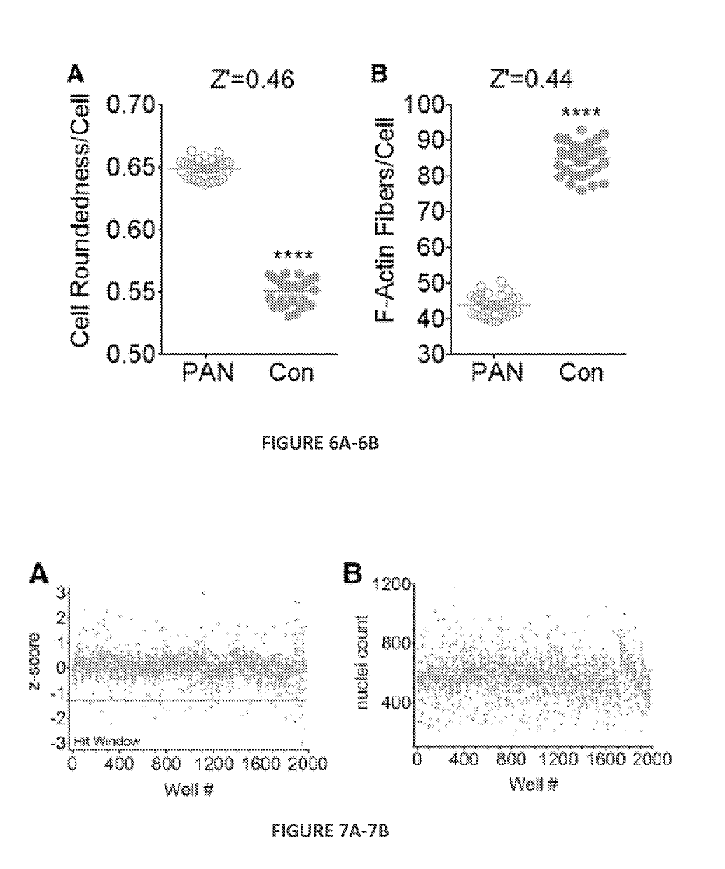

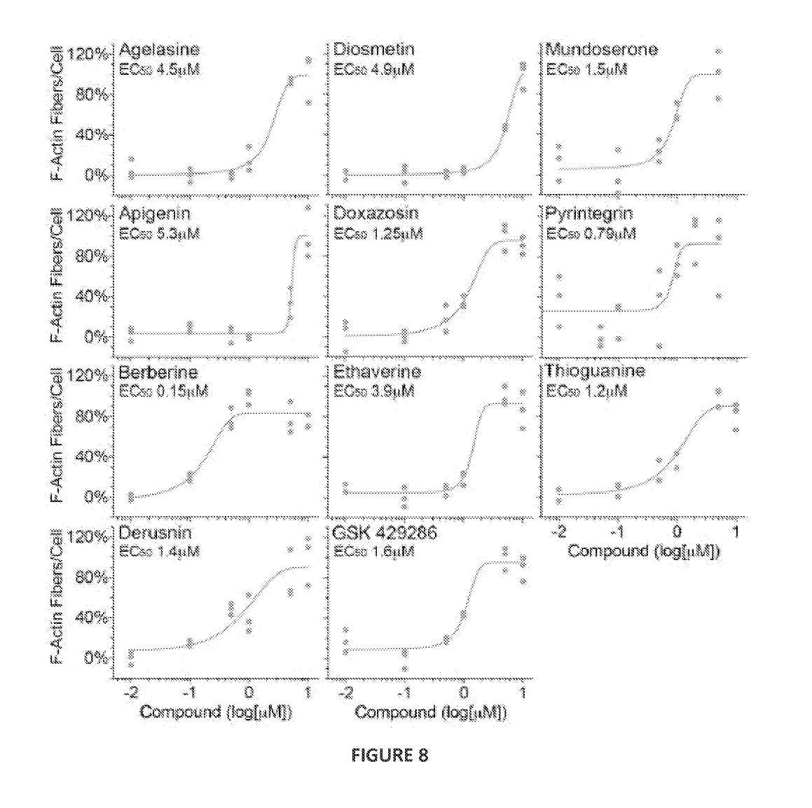

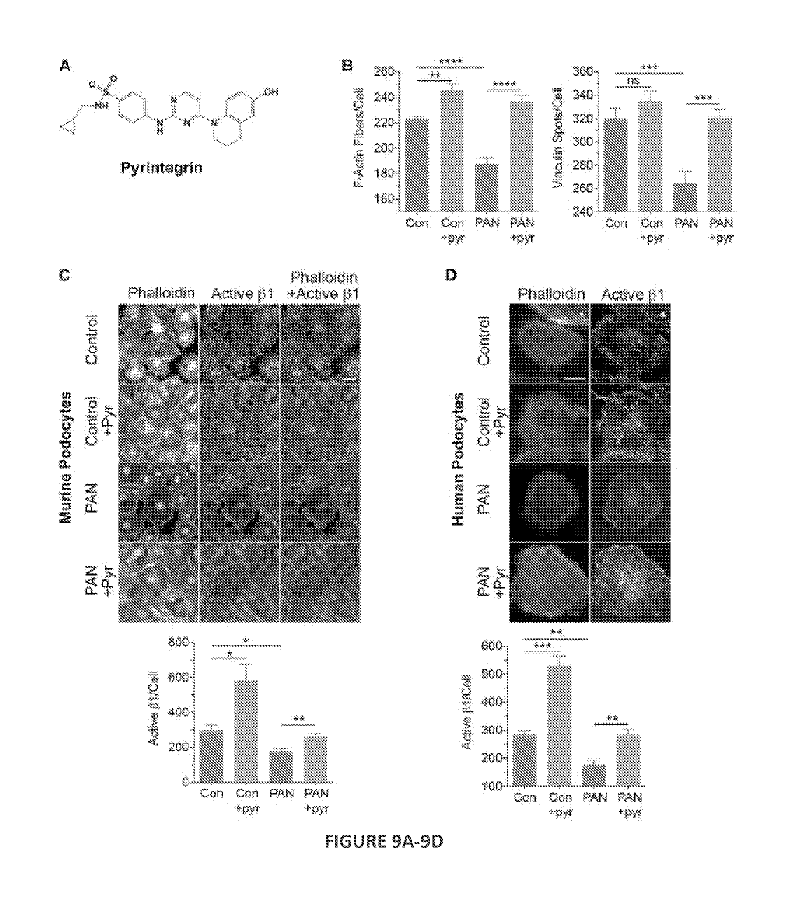

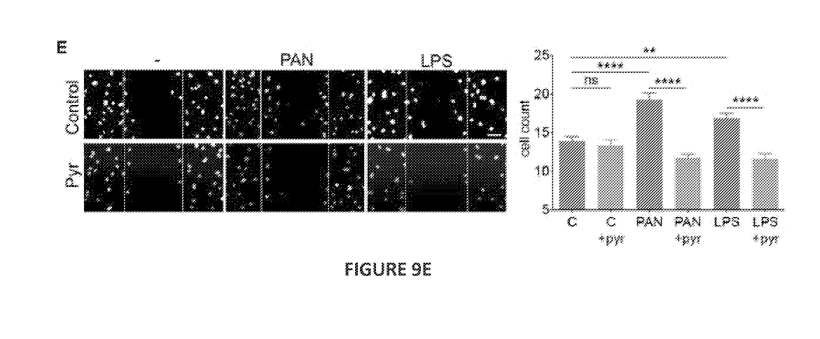

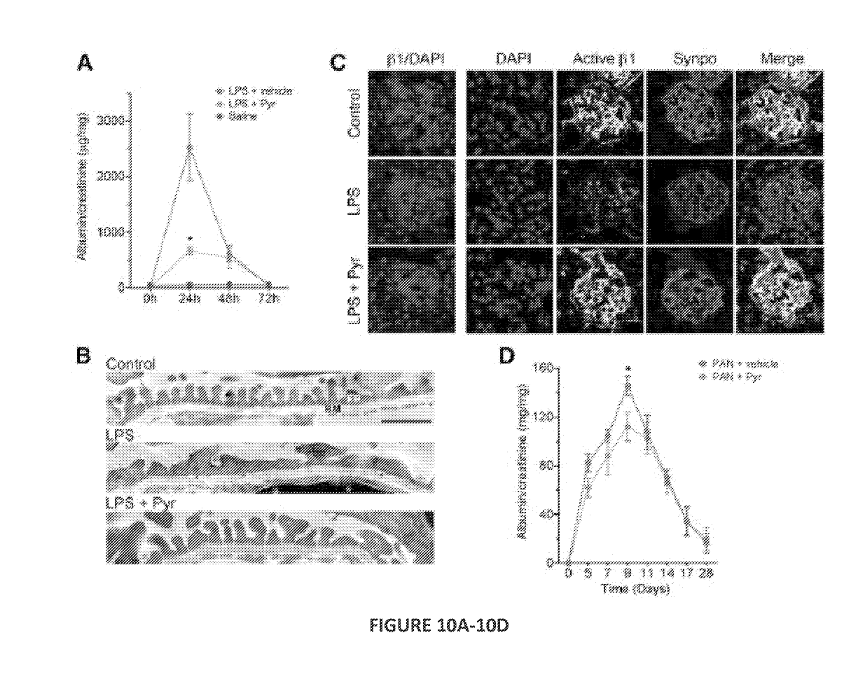

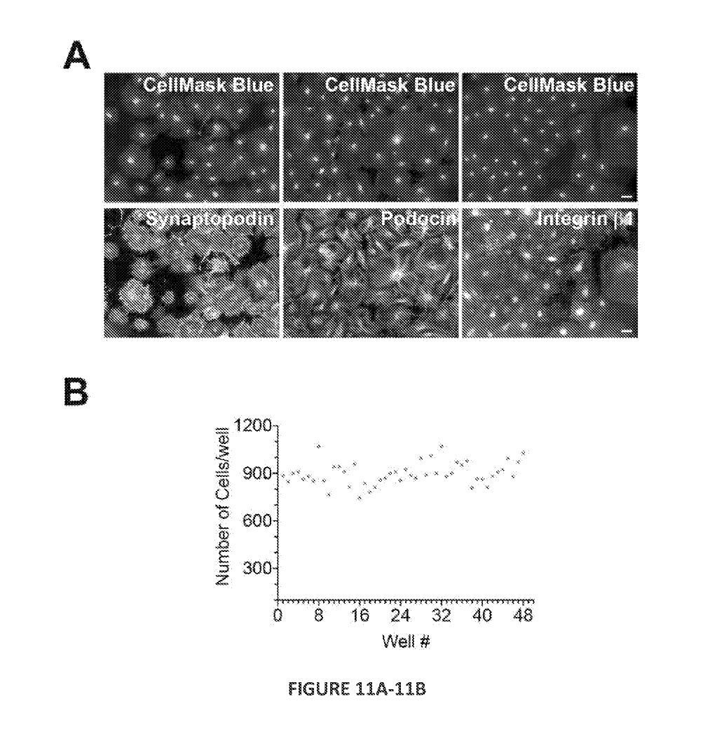

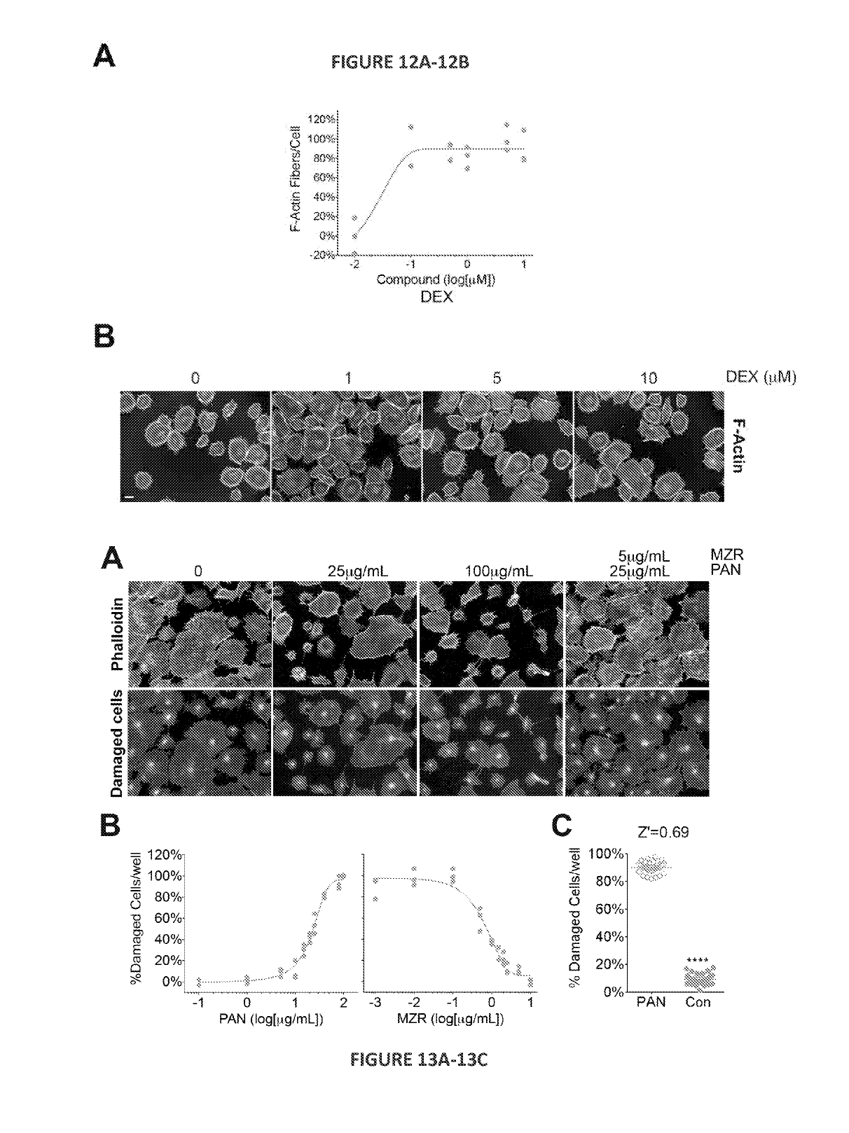

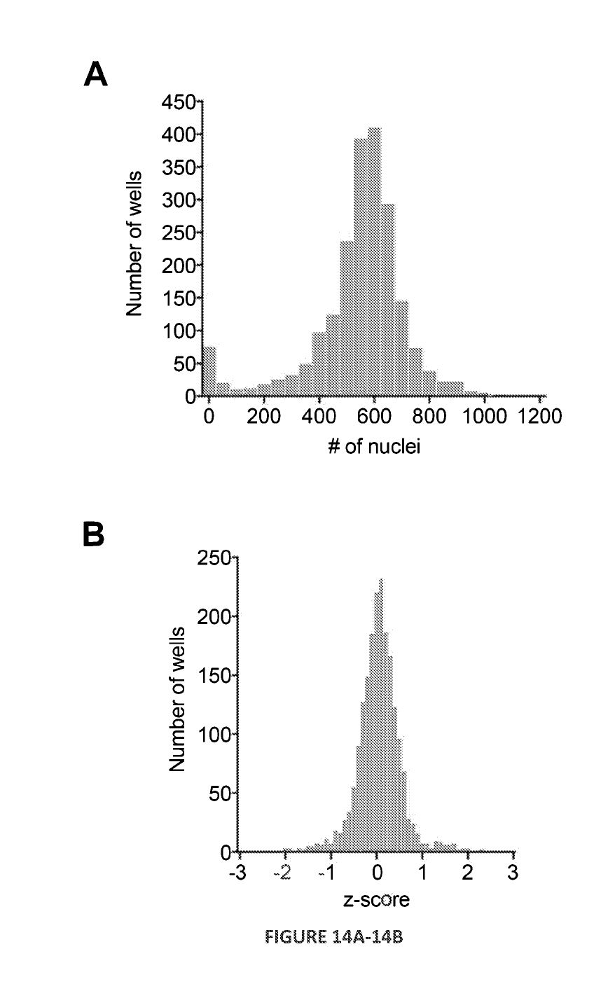

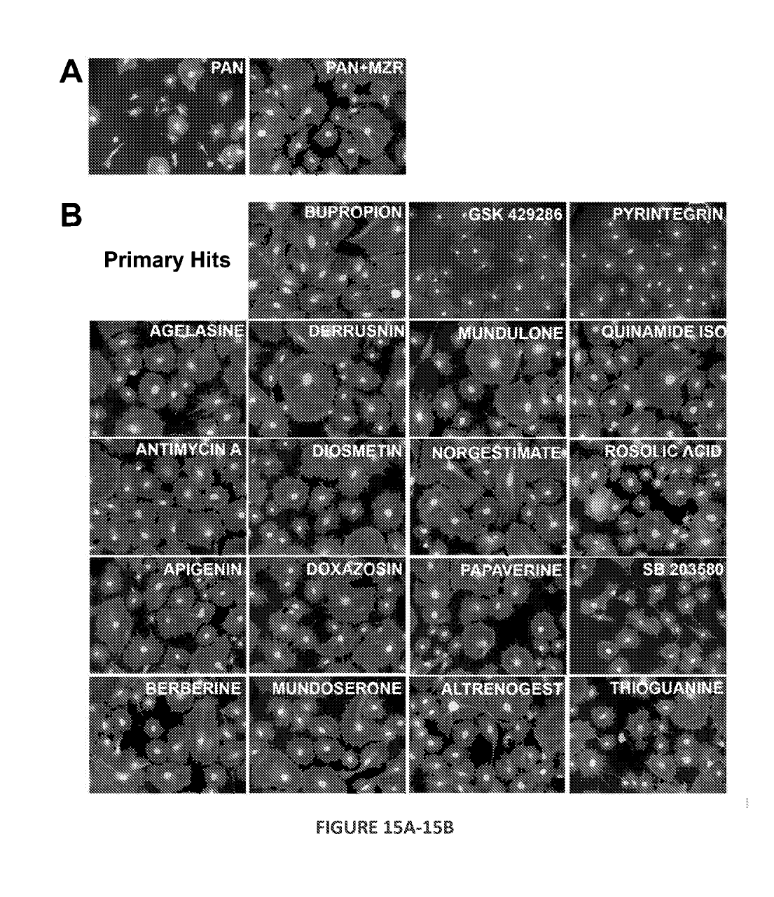

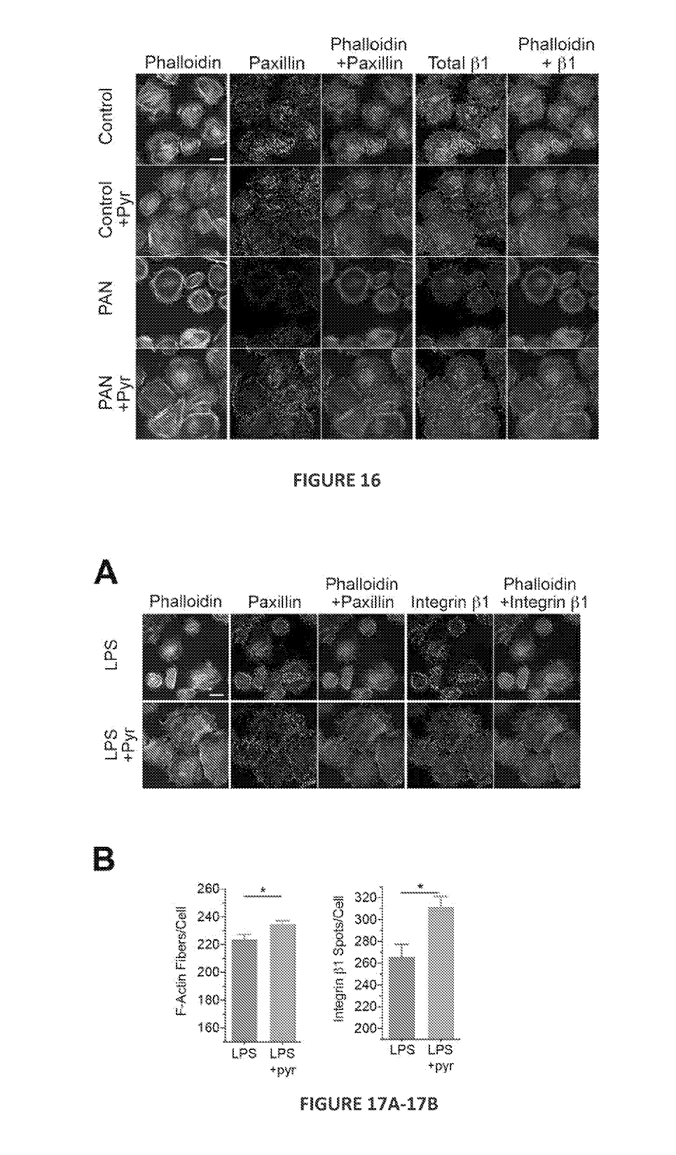

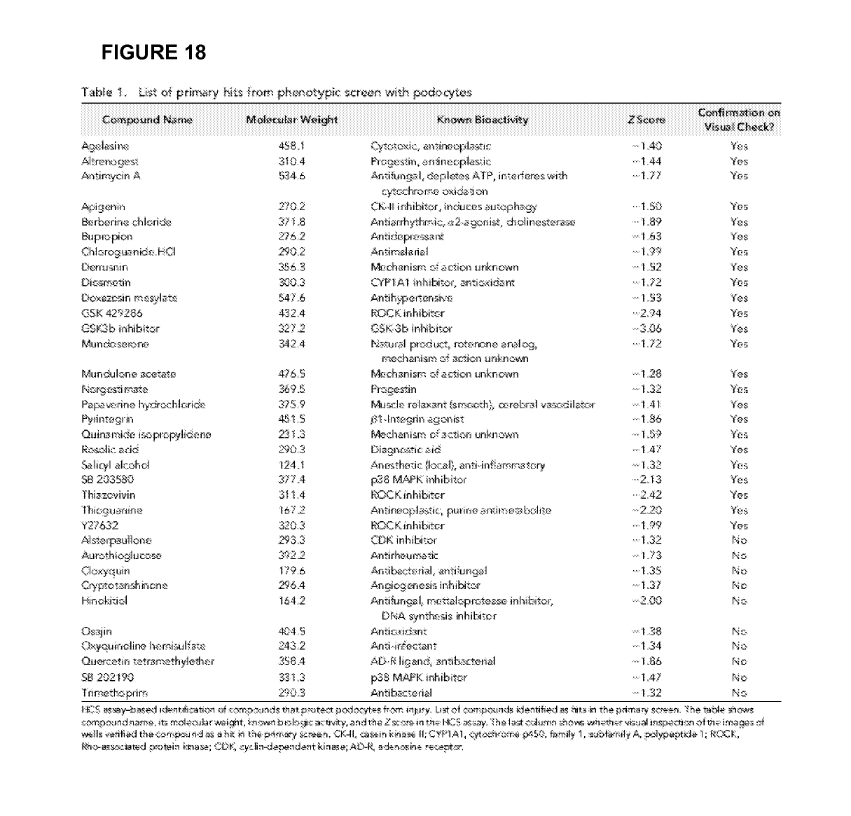

Mizoribine (MZR) is known in the literature to prevent PAN-induced podocyte damage. FIG. 2 shows that MZR co-treatment dose-dependently prevents podocyte damage, as quantitated using a variety of parameters in the HCS assay. Differentiated murine podocytes were cultured in multiwall plates, were treated with PAN (15-30 ug/mL) and were co-treated with an increasing dose of MZR and incubated at 37° C. for 48 hours. Podocyte damage was assessed at 48 h. A-D. Dose-response curves using various parameters show a dose-dependent change and MZR-induced podocyte protection. Each data point represents mean±SE of 3-6 wells and is representative of 2-3 independent assays.

FIG. 3A-3C. HCS assay provides robust differences between PAN-damaged and MZR-rescued cells using a multi-parameter algorithm. FIG. 3 shows that podocyte health and damage can be effectively quantitated using a combination of cellular parameters that are measured by OPERA and Columbus. A machine-learning algorithm was developed using a combination of parameters (and different weightages for those parameters) to segregate cells in each well into three different populations. The cellular populations were then classified into “percent healthy” or “percent damaged” and plotted in this dose-response series. A. Dose-response curve using “percent damaged” algorithm shows a dose-dependent increase in podocyte damage with PAN. Each data point represents mean±SE of 3-6 wells and is representative of 2-3 independent assays. B. Dose-response curve using “percent damaged” algorithm shows a dose-dependent decrease in podocyte damage with PAN and MZR co-treatment. Each data point represents mean±SE of 3-6 wells and is representative of 2-3 independent assays. C. Z-prime curve between PAN and PAN+MZR co-treatments on a single plate shows a clear and robust separation between the percent damaged cells under each treatment condition and a Z-prime value of 0.68, which is robust for high-throughput screening applications. Each data point is from a single well and mean±SE of approximately 48 wells and is representative of 2-3 independent assays.

FIG. 4A-4P. HCS assay based identification of compounds that protect podocytes from injury. FIG. 4 shows that podocyte health and damage can be effectively quantitated using a combination of cellular parameters that are measured by OPERA and Columbus. A machine-learning algorithm was developed using a combination of parameters (and different weightages for those parameters) to segregate cells in each well into three different populations. The cellular populations were then classified into “percent healthy” or “percent damaged” and plotted in this dose-response series. A-P. Dose-response curves showing dose-dependent decrease in PAN-induced podocyte damage upon co-treatment with each of the compounds shown in the presence of 30 ug/ml PAN. Compound names are shown above each graph. Each data point represents mean±SE of 3-6 wells and is representative of 1-3 independent assays.

FIG. 5A-5C. High content imaging and automated analysis can be used to design a podocyte phenotypic assay. 5A Schematic of the assay design. 5B Schematic of a well of a 96-well optical plate and the layout of various imaging frames that were typically captured using an HCS imaging system. 5C HCS-based image analysis of a podocyte phenotypic assay.

FIG. 6A-6B. Podocyte phenotypic assay has low variability. Graphs showing analysis of assay variability of the newly developed podocyte cell-based assay using automated microscope-based quantification of (A) cell roundness (morphology) or (B) F-actin fibers. Each graph shows quantified per-cell parameters from individual wells per condition (n=500-1000 cells per well) and the calculated means±95% confidence intervals across 30 wells and is representative of at least two independent assays. The calculated Z′ value between cells treated with PAN alone (damaged) or medical alone (con) is also shown. In both types of analyses, z′ value is >0.44. ****P<0.0001.

FIG. 7A-7B. HCS assay using a library of bioactive compounds identifies novel podocyte-protective agents. (A) A graph showing results from the screening of a chemical library using the podocyte HCS assay. Podocytes in 96-well optical plates were treated with PAN (16 μg/ml) at 37° C. for 48 hours, and 2121 pharmacologically active compounds were screened against it in the phenotypic assay. Each data point represents measured activity of each compound on cell roundness phenotype and its calculated Z score (Z score=0 means inactive compound, a positive value represents compounds that produce worse phenotypes, and a negative Z score represents active compounds). A dotted line marks the Z-score threshold of ≦−1.28 that was applied to obtain 34 primary hits in the hit window. Visual confirmation led to a final list of 24 active compounds. (B) A graph showing the nuclei count in each of the assay wells from the primary screen presented in A. Compounds resulting in a nuclei count of <200 were removed from the analyses as potentially cytotoxic agents.

FIG. 8. Independent assays with select primary hist show dose-dependent protection of podocytes, confirming their validity as a hit. Graphs showing does-dependent protection of podocytes from PAN injury by compounds identified in the primary screen. The name of each of the selected active compounds is shown on each graph. Podocytes in 96-well optical plates were incubated with PAN (16 μg/ml) at 37° C. for 48 hours in the presence of increasing doses of each of the selected compounds, and the cellular damage was quantified by measuring change in F-actin fibers per cell. F-actin fiber counts from podocytes treated with PAN alone (16 μg/ml; negative control) or PAN (16 μg/ml) and MZR (5 μg/ml; positive control) were set at 0% and 100%, respectively, to normalize the data. Each data point on the graphs represents normalized per-cell F-actin fiber count from individual wells per condition (n=500-1000 cells per well). Curve fitting was used to calculate apparent half-maximal effective concentration (EC₅₀) values (green line) and shows a dose-dependent protection of podocytes from PAN damage by each compound.

FIG. 9A-9E. An An β1-integrin agonist pyrintegrin (pyr) protects podocytes from damage. (A) The published chemical structure of pyr. (Xu et al. PNAS, 2010.) (B-D) Pyr protects podocytes from PAN-induced loss of F-actin fibers and focal adhesions and enhances active β1-integrin levels. (B) Graphs showing the effect of pyr on the number of F-actin fibers per cell and the number of vinculin spots per cell under each treatment condition. Data shown are means 6 SEMs per cell from three replicate wells (n=500-1000 cells per well). **P, 0.01; ***P, 0.001; ****P, 0.001. (C) Representative fluorescence images of murine podocytes after various treatments and after staining with phalloidin and antiactive β1 antibody. Two-color costained images show cells stained with phalloidin (red) and anti-active β1 antibody 9EG7 (green). Images were acquired using the Opera HCS System. The graph (lower panel) shows quantification of the active β1-integrin spot intensity per cell under each treatment condition. Data shown are means±SEMs per cell from four to five replicate wells (n=500-1000 cells per well). Scale bar, 50 μm. *P, 0.01; **P, 0.001. (D) Representative fluorescence images of human podocytes after various treatments as shown. Images show cells stained with phalloidin (red) and anti-active β1-integrin antibody 12G10 (green). Images were acquired using the Opera HCS System. The graph (lower panel) shows quantification of the active β1-integrin spots per cell under each treatment condition. Data shown are medians±SEMs per cell from four to five replicate wells (n=500-1000 cells per well). Scale bar, 50 μm. **P, 0.01; ***P, 0.001. (E) Pyr reduces injury-mediated increase in podocyte migration in a scratch wound-healing assay. Representative images (left panel) and a bar graph (right panel) showing confocal microscopy-based quantitation of the number of migrating podocytes in a scratch wound-healing assay Lines represent the wound margins at the beginning of the experiment (0 hours). The data presented in the graph are plotted as means±SEMs (n≧15). C, control. Scale bar, 50 μm. **P<0.01; ****P<0.001.

FIG. 10A-10D. β1-Integrin agonist protects animals against proteinuria. (A-C) Pyrintegrin (Pyr) protects mice from LPS-induced proteinuria. (A) Graph showing the ratio of albumin to creatinine in the urine of mice at the indicated time points after LPS administration and treatment with either Pyr (LPS+Pyr) or vehicle alone (LPS+vehicle). Control animals were administered saline alone. Data shown are means±SEMs (n=6-13 per group). *P<0.05. (B) Pyr protects against LPS-induced podocyte FP effacement. Representative electron microscopy image of mouse glomeruli treated with saline alone (control), LPS and vehicle (LPS), or LPS and Pyr (LPS+Pyr). *Effaced FPs in LPS-treated sections. BM, basement membrane. Scale bar, 1 μm. (C) Pyr preserves the level of active β1-integrin expression in the glomeruli. Representative confocal microscopy images of immunofluorescently labeled glomeruli from animals 24 hours after LPS administration. Frozen kidney sections from animals treated with saline alone (control), LPS and vehicle (LPS) or LPS and Pyr (LPS+Pyr) were imaged after staining with 4′,6-diamidino-2-phenylindole (DAPI) and antibodies against total β1 integrin (β1/DAPI), active β1 integrin (active β1), or synaptopodin (synpo). Merged active β1 and synpo channels are also shown (merge column). Scale bar, 25 μm. (D) Pyr protects rats from PAN-induced nephropathy. Graph showing the ratio of albumin to creatinine in the urine of rats at the indicated time points after PAN administration and treatment with either Pyr (PAN+Pyr) or vehicle alone (PAN+vehicle). Data shown are means±SEMs (n=4-6 per group). *P<0.05.

FIG. 11A-11B. Cell cultured in 96-well optical plates for HCS assays express typical markers of healthy podocytes. Cell cultured in 96-well optical plates for HCS assays express typical markers of healthy podocytes. 11A. Representative images of single frames from a 96-well plate showing podocytes cultured in optical plates and stained with HCS CellMask blue (top panels) and antibodies against synaptopodin, podocin or integrin β1 (bottom panels). Images were acquired using PerkinElmer Opera high-content screening microscope. Scale bar, 50 μm. 11B. A graph showing the quantification of the number of nuclei in each of the 48 wells of a plate. Podocytes were cultured in 48 replica wells of a 96-well optical plate, as described in the methods section. Cells in each well were stained with CellMask Blue, which also stains the nuclei. Wells were imaged using Opera HCS system and nuclei in each well were automatically counted. The graph shows that podocytes cultured in multi-well plates show low well-to-well variability in cell number.

FIG. 12A-12B. Dexamethasone (DEX) dose-dependently protects podocyte from PAN injury. Dexamethasone (DEX) dose-dependently protects podocyte from PAN injury. Podocytes in 96-well optical plates were co-treated with PAN (30 μg/mL) and an increasing concentration of DEX at 37° C. for 48 h and the cellular damage was assessed using HCS system. 12A. A dose-response curve showing the protective effects of increasing concentration of DEX on F-actin fiber count per cell. Data shown are means±the standard error of the mean (SEM) per cell from a single assay well (n=500-1000 cells), performed in three replicate wells. 12B. Representative fluorescence images of cells co-treated PAN (30 μg/mL) and an increasing dose of DEX (as shown) and stained with CellMask Blue and phalloidin are shown. Scale bar, 50 μm.

FIG. 13A-13C. HCS assay using a multi-parameter algorithm also shows low variability. HCS assay using a multi-parameter algorithm shows low variability. Podocyte health and damage can be quantified using a combination of cellular parameters that were measured by Opera and Columbus. A machine-learning algorithm was used to segregate cells in each well into “percent healthy” or “percent damaged” and plotted in this dose-response series. 13A. Representative images after processing with a multiparameter algorithm showing phalloidin stained cells (top panel) after treatment with various doses of PAN and PAN+MZR, as indicated. Cells automatically identified by the machine-learning algorithm as “damaged” in each image frame are colored red in the bottom panel. Scale bar, 50 μm. 13B. Dose-response curves using “percent damaged” algorithm showing a dose-dependent increase in podocyte damage with PAN (left) or a dose-dependent decrease in podocyte damage with PAN and MZR co-treatment (right). Each data point represents results from three replicated wells. 13C. A graph showing analysis of assay variability of the newly developed podocyte cell-based assay and analyzed using the automated multi-parameter algorithm for “percent damaged” cells under each condition (n=500-1000 cells/well) and the calculated mean±95% confidence intervals across 30 wells. The calculated Z′-value between cells treated with PAN alone (damaged) or healthy cells (control) is also shown. **** p<0.0001.

FIG. 14A-14B. Chemical screening data shows bell shaped curve. 14A. A frequency plot showing the distribution of total nuclei count per assay well (x-axis) from the primary assay (bin width=50) versus the number of compounds or wells (frequency) in each bin of the nuclei count on y-axis. 14B. A frequency plot showing the distribution of z-score values (bin width=0.1) from the primary assay versus the number of compounds or wells (frequency) in each bin of the z-score on y-axis.

FIG. 15A-15B. Images of cells from the assay wells of the primary screen. Representative CellMask Blue stained fluorescence images from primary screen showing (15A) control cells treated with PAN alone or PAN+MZR (as shown), or (15B) cells co-treated with PAN and the indicated compound. Scale bar, 50 μm.

FIG. 16. Images of murine podocytes showing that pyrintegrin (pyr) protects podocytes from PAN-induced damage. Podocytes in 96-well optical plates were cultured at 37° C. for 48 h in the absence (Control, Con) or presence of PAN (30 μg/mL) and co-treated with vehicle (DMSO, 1%) or pyrintegrin (pyr) (1 μM). The cellular damage was assessed after staining the cells with phalloidin, anti-paxillin and anti-integrin β1 antibodies and quantifying various cellular phenotypes using the HCS system. The panel here shows representative fluorescence images of the cells. Two color co-stained images show cells stained with phalloidin (red) and anti-paxillin antibody (green) (central panels) or phalloidin (red) and anti-integrin β1 antibody (green) (right panels). Images were acquired using a confocal microscope. Scale bar, 50 μm.

FIG. 17A-17B. Pyrintegrin (pyr) protects podocytes from LPS-induced loss of F-actin fibers and enhances integrin β1. Podocytes in 96-well optical plates were cultured at 37° C. for 48 h in presence of LPS (100 μg/mL) and co-treated with vehicle (DMSO, 1%) or pyr (1 μM) and the cellular damage was assessed after staining the cells with phalloidin, anti-paxillin or anti-vincullin and anti-β1 antibodies and quantifying various cellular phenotypes using the HCS system. 17A. Representative fluorescence images of murine podocytes after various treatments, as described in panel 17B and shown on the left side of the images, and after staining with phalloidin, anti-paxillin or anti-β1 antibodies. Two color co-stained images show cells stained with phalloidin (red) and anti-paxillin antibody (green) (central panels) or phalloidin (red) and anti-β1 antibody (green) (right panels). Images were acquired using the Opera HCS system. Scale bar, 50 μm. 17B. Graphs showing the effect of pyrintegrin on the number of F-actin fibers per cell and the integrin β1 spots per cell under each treatment condition are presented. Data shown are means±SEM per cell from three replicate wells (n=500-1000 cells/well). *p<0.05.

FIG. 18. Table 1: List of primary hits from phenotypic screen with podocytes.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings.

Podocytes represent a crucial target for therapeutic interventions in Focal segmental glomerulosclerosis (FSGS) and other proteinuric glomerular diseases. However, current methods are inadequate for podocyte targeted drug discovery. Here, we aim to develop novel, podocyte cell-based assays for use in a drug discovery environment and use these assays to identify new therapeutic leads.

Normal actin cytoskeletal machinery is essential for maintaining podocytes in a healthy state in vivo and in vitro. A high content screening (HCS) based methodology was developed to quantitatively measure actin cytoskeleton and other cellular features in healthy podocytes. A computational methodology to quantitatively measure changes in podocytes after treatment with an increasing dose of podocyte damaging agent puromycin aminonucleoside (PAN) was also developed. Cellular classification based on various phenotypic feature allowed a robust determination of PAN induced changes in cells to separate the damaged and healthy cell populations and the data was used to generate a percent healthy cell statistic per well. Treatment with known podocyte-protective agents, such as mizouribine, showed a quantitative, dose-dependent protection of podocytes from damage in the HCS assay. A chemical library was also examined in the HCS assay to identify compounds that could potentially protect podocytes PAN-induced damage. The HCS assay quantitatively defined dose-dependent changes in a variety of podocyte markers and phenotypes, including changes in cellular morphology, F-actin and focal adhesion markers. The HCS assay is robust and has a Z-prime factor >0.5, which makes it suitable for use in a high-throughput screening environment. Screening with a small chemical library identified a number of novel compounds that protected podocytes from PAN-induced damage (˜1% hit rate).

A high-throughput assay for unbiased quantification of changes in podocyte health that has wide applicability is described herein. Using this HCS assay, we have identified a plurality of compounds that show podocyte protection in a dose-dependent fashion. The new agents could serve as therapeutics for treating various kidney diseases.

The invention generally relates to methods of culturing mammalian podocytes in vitro. The cultures can be used for drug screening (such as medium or high throughput drug screening), for studying molecular pathways involved in glomerular diseases and for assessment of patient samples. The invention also provides methods for analyzing the viability of podocytes in a cell culture, using parameters such as specific measurements of the actin cytoskeleton, focal adhesions, and morphology. The invention also provides a number of chemical agents that either prevent or treat podocyte injury or both. The invention provides chemical agents as therapeutics for treating kidney diseases.

Screening Assays

In some embodiments, the invention provides methods of screening libraries or agents to identify agents for use in treating kidney disease or for protecting podocytes from injury. In some embodiments, the screening methods include adding a test agent to a podocyte culture and incubating the agent with the culture. The method may also include comparing the test agent to a podocyte damaging control and/or a podocyte protecting control. In some embodiments, the screening assay may identify a kidney disease causing agent in a patient's sample. In yet other embodiments, the screening assay may identify structural or functional defects in a patient. The screening assay may measure one or more of the following but is not limited thereto: cell morphology, cell roundedness, F-actin intensity, number of focal adhesions, intensity or size of focal adhesions, length of actin fibers, size of actin fibers, percent damaged or percent healthy cells, number of actin fibers per cell number of synaptopodin fibers per cell and β1 integrin levels. In some embodiments, the screening assay may measure one or more of the following but is not limited thereto: active integrin β1, integrin αvβ3, active integrin β3, integrin β5, active integrin β5, desmin, podocin, synaptopodin, WT1, podocalyxin, nephrin, neph 1, and CD2 associated protein (CD2AP).

Measurement of the endpoints of the screening assays may be accomplished using any method known to one skilled in the art. In some embodiments, measurements may be obtained using microscopy. In some embodiments, light, confocal or electron microscopy may be used but not limited thereto. Methods for measuring protein expression include, but are not limited to Western blot, immunoprecipitation, immunohistochemistry, Enzyme-linked immunosorbent assay (ELISA), Radio Immuno Assay (RIA), radioreceptor assay, proteomics methods (such as mass spectrometry), or quantitative immunostaining methods. Methods for measuring nucleic acid expression or levels may be any techniques known to one skilled in the art.

Methods of Treatment

In various embodiments, the invention provides methods for treatment of a patient having a kidney disease. In some embodiments, the invention provides a method of protecting podocytes from injury. The methods generally comprise administration of an agent to treat the kidney disease or to protect podocytes from injury. The method may include administration of one or more agents in a therapeutically effective amount.

“Treating”, “treat”, or “treatment” within the context of the instant invention, means an alleviation of symptoms associated with a disorder or disease, or halt of further progression or worsening of those symptoms, or prevention or prophylaxis of the disease or disorder. For example, within the context of this invention, successful treatment may include prevention of a kidney disease, an alleviation of symptoms related to kidney disease or a halting in the progression of a disease such as a kidney disease.

In some embodiments, the inhibitor is a chemical compound, natural or synthetic, in particular an organic or inorganic molecule of plant, bacterial, viral, animal, eukaryotic, synthetic or semisynthetic origin, capable of treating a kidney disease. Non-limiting examples of a chemical compounds that may be used to treat kidney disease or to protect podocytes from injury may include one or more of the following Pyrintegrin; Kenpoullon; Kinetin; Apigenin; Agelasine; Berberine; Derusnin; Ethaverine; Diosmetin; Doxazocin; Thioguanidine; P38 inhibitor (SB239063); FR 180204; Mundeserone; Y27632; Alk5 Inhibitor2; and GSK429286.

As used herein, the term “pharmaceutically acceptable salts” include those salts formed, for example, as acid addition salts, preferably with organic or inorganic acids, from compounds with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, malonic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 4-toluenesulfonic acid, 2-naphthalenesulfonic acid, 1,5-naphthalene-disulfonic acid, 2- or 3-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

The compounds may be used alone or in compositions together with a pharmaceutically acceptable carrier or excipient. Pharmaceutical compositions of the present invention comprise a therapeutically effective amount of a compound formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds may be administered to humans and other animals orally, parenterally, sublingually, by aerosolization or inhalation spray, rectally, intracisternally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1-5 μm. Predominantly in this application means that at least 70% but preferably more than 90% of all generated aerosol particles are within 1-5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic-Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

A compound can be administered alone or in combination with another agent, possible combination therapy taking the form of fixed combinations or the administration of a compound and another agent being staggered or given independently of one another. Long-term therapy is equally possible as is adjuvant therapy in the context of other treatment strategies, as described above. Other possible treatments are therapy to maintain the patient's status after kidney disease, or even preventive therapy, for example in patients at risk.

Effective amounts of the compounds of the invention generally include any amount sufficient to detectably inhibit the presence or progression of kidney disease, or by detecting an inhibition or alleviation of symptoms of kidney disease. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

According to the methods of treatment of the present invention, kidney disease is reduced or prevented in a patient such as a human or lower mammal by administering to the patient an amount of an agent, in such amounts and for such time as is necessary to achieve the desired result. An “amount that is effective to inhibit the presence or progression of kidney disease” of a compound refers to a sufficient amount of the compound to treat kidney disease, at a reasonable benefit/risk ratio applicable to any medical treatment.

It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The dose of the agent for treatment of kidney disease or for protecting podocytes to be administered alone or in combination therapy warm-blooded animals, for example humans, is preferably from approximately 0.01 mg/kg to approximately 1000 mg/kg, more preferably from approximately 1 mg/kg to approximately 100 mg/kg, per day, divided preferably into 1 to 3 single doses which may, for example, be of the same size. Usually children receive half of the adult dose, and thus the preferential dose range for the inhibitor in children is 0.5 mg/kg to approximately 500 mg/kg, per day, divided preferably into 1 to 3 single doses that may be of the same size.

Diagnostic Assays

In some aspects, the present invention relates to methods of detecting the presence of one or more diagnostic markers in a sample (e.g. a biological sample from a patient) or detecting the presence of a disease causing agent in a sample. A variety of screening methods known to one of skill in the art may be used to detect the presence of the marker in the sample including DNA, RNA and protein detection. In some embodiments, the detection system will use fluorescence microscopy as a read out for the diagnostic assays. The techniques described below can be used to determine the presence or absence of a defect in structure or function of podocytes in a sample obtained from a patient relative to a sample obtained from a control subject sample. The techniques described below can also be used to determine the presence or absence of disease causing agents in a sample such as blood, serum or urine. In some embodiments, the sample may be a podocyte sample from the patient and assayed for cell health and damage using one or more of the following markers, including, but not limited to cell roundedness, focal adhesions per cell, actin fibers per cell, synaptopodin fibers per cell and β1 integrin levels. In some embodiments, the sample may be a patient sample that is added to a podocyte culture and incubated with the culture. Following incubation, the podocyte culture may be assayed for cell health and damage using one or more of the following markers, including, but not limited to cell roundedness, focal adhesions per cell, actin fibers per cell, synaptopodin fibers per cell and β1 integrin levels. Examination of cell health or damage markers in a patient assists the physician in determining a treatment protocol for the patient.

In some embodiments, practice of the present invention will employ, unless otherwise indicated, conventional techniques of molecular biology, immunology, microbiology, cell biology and recombinant DNA, which are within the skill of the art. See e.g., Sambrook, Fritsch and Maniatis, MOLECULAR CLONING: A LABORATORY MANUAL, (Current Edition); CURRENT PROTOCOLS IN MOLECULAR BIOLOGY (F. M. Ausubel et al. eds., (Current Edition)); the series METHODS IN ENZYMOLOGY (Academic Press, Inc.): PCR 2: A PRACTICAL APPROACH (Current Edition) ANTIBODIES, A LABORATORY MANUAL and ANIMAL CELL CULTURE (R. I. Freshney, ed. (Current Edition)). DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); Fundamental Virology, 2nd Edition, vol. I & II (B. N. Fields and D. M. Knipe, eds.)

Experimental Methods

Reagents and Antibodies

Fetal bovine serum (FBS) was purchased from Hyclone and all other cell culture reagents were from Life Technologies (Carlsbad, Calif.). Alexa Flour568-labeled phalloidin and HCS CellMask blue were obtained from Life Technologies (Carlsbad, Calif.), anti-paxillin antibody clone Y113 was from Millipore (Billerica, Mass.), polyclonal goat anti-Synaptopodin (synpo) antibody P-19 (SC-21537) was from Santa Cruz (Dallas, Tex.), anti-vinculin antibody clone hvin was from Sigma-Aldrich (St. Louis, Mo.), anti-podocin antibody was from Santa Cruz (Dallas, Tex.), hamster anti-total β1 integrin antibody HMB1-1 was from BioLegend (San Diego, Calif.), rat anti-mouse active integrin β1 antibody 9EG7¹ was from BD Biosciences (San Jose, Calif.) and mouse anti-human active integrin β1 antibody 12G10² was from Abcam (Cambridge, Mass.). Rat tail Collagen I, puromycin aminonucleoside (PAN), lipopolysaccharide (LPS), mizoribine (MZR) and dexamethasone (DEX) were from Sigma-Aldrich (St. Louis, Mo.). Pyrintegrin (pyr) was from EMD Millipore (Billerica, Mass.).

Animals

Animal care and procedures were approved by the Institutional Animal Care and Use Committee (IACUC) and were performed in accordance with institutional guidelines. The C57BL/6J (B6) wild-type mice and Sprague-Dawley (SD) wild type rats were purchased from The Jackson Laboratory.

Cell Culture

Conditionally immortalized human and murine podocytes were used in a majority of the assays. Human and murine podocytes were cultured as have been described previously (Saleem et al., JASN 13, 630-638 (2002); Mundel, Reiser et al., Experimental Cell Research, 236, p 248 (1997)). For example, murine podocytes derived from the mouse line H-2Kb-tsA58 were cultured under permissive conditions at 33° C. on rat-tail collagen coated dishes in RPMI medium supplemented with 10% heat inactivated FBS, penicillin and streptomycin (100 U/ml), and 10 U/ml recombinant mouse γ-interferon. Cells were then trypsinized and re-plated for differentiation at 37° C. in medium without γ-interferon. The cells were differentiated for 0 to 7 days and then reseeded to rat-tail collagen coated multi-well plates at a density of 1500/well or 500/well for 96 and 384 well plates, respectively. Cells were then further differentiated for another 5-14 days in the multi-well plates for subsequent experimentation with various reagents prior to fixation and analyses.

In some embodiments, podocytes were cultured at 37° C. for the first 7 days of differentiation in large tissue culture flasks and subsequently reseeded and further cultured at 37° C. in the 96-well assay plates for the next 4-6 days to provide low well-to-well variability in cell number in the assay plates and healthy podocytes expressing specific and well characterized podocyte markers synaptopodin and podocin. A schematic of the assay design is shown in FIG. 5A.

Primary podocytes may also be used in the assays described herein. Methods for isolating and culturing primary podocytes may be found in Kabgani et al., Primary Cultures of Glomerular Parietal Epithelial Cells or Podocytes with Proven Origin, Plos One, PLoS ONE (2012) 7(4): e34907. doi:10.1371/journal.pone.0034907. Primary podocytes cultured for use in the HCS assay were cultured in Dulbeco's Modified Eagle Medium (DMEM) with L-glutamine supplemented with 10% fetal bovine serum (FBS), and with 100 U/mL Penicillin-Streptomycin. Podocytes were partially differentiated at 37 degrees for 7 days and then reseeded into multi-well plates where they were then used for experiments after 3-7 days, as described above.

High-Throughput High-Content Screening Using a High-Content Fluorescence Microscopy System (OPERA)

Differentiated podocytes were cultured in 96-well or 384-well multiwall optical plates (PerkinElmer, Boston, Mass.). In some embodiments, differentiated podocytes were treated with various amounts of puromycin aminonucleoside (PAN), mizorbine (MZR), or LPS, Adriamycin and other agents. In some embodiments, the cells were co-treated with chemical and other agents (such as mizouribine) to prevent damage from damaging agents (such as PAN). In other embodiments, the rescue agents were added 0-48 h after the addition of injury causing agents. In some embodiments, patient samples such as blood, serum, urine or other samples were added to differentiated podocytes to identify disease causing agents. In some embodiments, primary, differentiated patient podocytes were cultured to identify defects in structure and/or function.

After culturing for the appropriate time and treatment or control, cultured podocytes in multi-well optical plates were rinsed with phosphate buffered saline (PBS) and subsequently fixed using a solution of paraformaldehyde and sucrose in PBS (at a final concentration of 4% paraformaldehyde and 2% sucrose in PBS) for 30 min at room temperature. Following fixation, the cells were permeabilization using a solution of 0.3% Triton X-100 in PBS for 15 min at room temperature. For visualization of filamentous-actin (F-Actin) in cells, cells were stained with a solution of Alexa fluor 568 phalloidin (Invitrogen), as previously described. HCS CellMask Blue (Invitrogen) was used as a fluorescent stain for visualizing nuclei, cytoplasm and the individual cell boundaries. Additional proteins that were fluorescently labeled and analyzed using appropriate primary mAbs include synaptopodin (synpo, primary mAb from Santa Cruz), Paxillin (primary mAb clone Y113 from Millipore) for quantitation of focal adhesions, beta1 integrin, podocin, WT1 nephrin, CD2-AP may also be used. Other proteins may also be used for labeling. Appropriate secondary antibodies (fluorescently labeled) were used for fluorescent visualizations. High throughput confocal microscopy was done using the Opera LX (Perkin Elmer) with filters and exposure times according to manufacturer's instructions and as described before. Images were used in subsequent image analysis for quantitation of a variety of cellular parameters. Assays were performed in replicate wells for assay robustness.

Image Analysis

Images for each frame of each well in multiple fluorescent channels were acquired using OPERA imaging system. Images were transferred to another disk and were subsequently analyzed and quantified (for various features) using Columbus 2.4.0 high content screening image data storage and Acapella version 2.0 data processing software (PerkinElmer). The HCS CellMask Blue stain (Invitrogen) was used to determine the number of nuclei in each frame and cell boundaries. In certain cases, multiple image frames for each well were joined or stitched together prior to image analyses.

Nuclei were usually detected using the “Find Nuclei” analysis module with method B and a common threshold of 0.90 and an area greater than 200 um². The cytoplasm was detected using the “Find Cytoplasm” analysis module using method B with a common threshold of 0.45 and an individual threshold of 0.15. Cellular morphology properties were calculated to determine cellular features, such as cell area and cell roundedness. F-actin fibers were sectioned from the image using the “Find Spots” analysis module using method B with detection sensitivity and a splitting coefficient of 0.4 and morphology and intensity properties were calculated using computational parameters in the program. Texture properties were calculated for F-actin using a scale of 1 pixel. To perform automated cell partitioning into multiple populations, based on a combination of various parameters analyzed in Columbus, we used a user-trained machine learning algorithm. User input was used to classify cellular population in a set of images, one image at a time, to visually define cells to be classified into an individual group. In certain cases, machine learning was used to classify cells into three populations. Subsequently, upon various rounds of training, an algorithm was developed to determine the best separation between populations using various combinations of measured cellular parameters. In one case, cells in each image were divided into three populations based upon the following linear coefficient values: −220.364 SER Bright, 210.842 SER ridge, −5.7e-5 Cell size um², 6.563 Relative spot to background intensity, 0.005688 Number of spots, 47.769 SER Saddle, −14.957 SER Valley, and 2.5079 Cell roundedness. Further subdivision between two of the selected populations was accomplished by placing a negative score for one and a positive score for the other using the following linear coefficient values: −0.00013529 Cell area um², 37.2151 SER Edge, 0.00891689 Number of spots, −74.81 SER Bright, −20.5945 SER Dark, −55.3194 SER Saddle. A combination of one or more population statistics was used to define percent healthy or percent damaged cells in each well, for use in a chemical screen, for example. (See FIGS. 5B and 5C illustrating the imaging frames and the image analysis.)

Chemical Library Screening

Screening of chemical libraries, which include the MicroSource Spectrum collection (MicroSource Discovery Systems, Inc., Gaylorsville, C T, 2000 compounds) and Enzo kinase inhibitor array (Enzo Life Sciences, Farmingdale, N.Y., 80 compounds), Sigma, Millipore, Tocris and Cayman (41 compounds), was performed using multiwall plates. Podocytes cultured in multiwall plates were used in these assays. 100 nL of each compound, also in multiwall plates (at a stock concentration of 1 mM to 10 mM in DMSO), was added to each well of a podocyte containing multiwall plate using the Perkin Elmer Janus automated workstation with the help of a 96-pin or 384-pin pin tool (100 nL, PerkinElmer). The compounds were added in the absence or presence of podocyte damaging agent (such as PAN). The plates containing the compounds were subsequently placed in tissue culture incubators and cells were further cultured for 1-72 h at 37° C. A compound that decreased PAN damaged was identified by quantifying the changes in at least one of the following parameters: cell morphology, cell roundedness, F-actin intensity, number of focal adhesions, intensity or size of focal adhesions, length of actin fibers, size of actin fibers, percent damaged or percent healthy cells etc. In certain cases, hits were validated by acquiring a fresh batch of the compound from commercial sources and performing dose-response curves. Images from the primary screen were quantified for cell morphology parameter and were normalized by calculating z-score (z) for each test compound, to compensate for any plate-to-plate variability. (Wiggins, Kidney International, 2007) z was calculated for each of the test compounds using the formula:

$$z = \frac{( x - μ )}{σ} ,$$

where x is the raw measured value with each compound, μ is the mean of all measured raw values across a plate and a is the standard deviation of the measurements. A z-score of 11.281, corresponding to 90% population under the curve, was used as a cutoff to identify primary hits in the assay.

Scratch, Wound-Healing Assay

The effect of identified novel compounds on podocyte migration was assessed using a classic scratch, wound-healing assay as described. (Mathieson, Nat Rev Nephrol, 2012.) Differentiated mouse podocytes per well were seeded overnight in type-I collagen coated wells of a 24 well plate. Each well was subsequently scratched using a sterile 200 μI pipette tip, washed and placed into fresh culture medium. Cells in wells were also treated with PAN, LPS, pyr or a combination, as described in the text. After 48 h, cells were fixed with 2% paraformaldehyde and stained with DAPI. Images of cells near scratched surfaces and their DAPI-stained nuclei were acquired using a 10× objective using a Zeiss Axio Observer D1 microscope (Carl Zeiss Group, Hartford, Conn.) and analyzed using ImageJ software (NIH). Images using transmitted light were also acquired at the beginning and the end of the experiment and were used to align images of pre- and post-treated cells for counting cells that had migrated into similar sized fields. The data shown represent the mean±SEM of three independent experiments.

LPS-Induced Proteinuria

The induction of proteinuria in female wild type C57BL/6J mice (n=6-13 per group) by LPS injection was performed as described. (Reiser et al., J Clin Invest, 2004; Yanagida-Asanuma et al., The American Journal of Pathology, 2007; Faul et al., Nat Med, 2008; Yu et al., The New England Journal of Medicine, 2013.) Briefly, 10 week old female mice were intraperitoneally injected with LPS (10 mg/kg, in saline). Control mice were injected with saline alone. Animals in the treatment groups were treated with vehicle or 10 mg/kg pyr (stock in DMSO) diluted in saline 2 h prior to the administration of LPS. Spot urine was collected from each group of animals for measurement of albuminuria as mg albumin per mg creatinine. (Faul et al., Nat Med, 2008) Results are expressed as mean±SEM. The mice were sacrificed and the efficacy of pyrintegrin was further analyzed by histochemical and immunofluorescence analyses.

PAN-Induced Nephropathy

The induction of nephropathy in male wild type SD rats (n=6 per group) by PAN injection was performed as described. (Reiser et al., J Clin Invest, 2004; Wei et al., Nature Medicine, 2008; Nakamura et al., Lab Invest, 1991.) Briefly, 5 week old male rats were intravenously injected with PAN (150 mg/kg, in water) and treatment with vehicle or pyrintegrin was started on the same day. Animals in the vehicle group were administered saline-based vehicle solution (3.3% DMSO and 5% Cremaphor EL) intraperitoneally daily for 14 days. Animals in the treatment group were treated with pyrintegrin (10 mg/kg, 3.3% DMSO and 5% Cremaphor EL) in the saline based solution daily for 14 days. Spot urine was collected from animals for measurement of albuminuria as mg albumin per mg creatinine. (Faul et al., Nat Med, 2008) Results are expressed as mean±SEM.

Light and Electron Microscopy

For light microscopy, freshly harvested mouse kidney tissue was embedded in OCT, snap frozen in liquid nitrogen and stored at −80° C. Tissue sections were cut and fixed in −20° C. acetone before immunofluorescence staining. Sections were blocked at room temperature for 1 h and incubated with the primary antibodies against total β1 integrin (hamster anti-mouse, BioLegend), active β1 integrin (Lentner et al., PNAS, 1993.) (rat anti-mouse antibody 9EG7, BD Biosciences) and synaptopodin (polyclonal goat, SC-21537, Santa Cruz) at 4° C. overnight. Slides were incubated with appropriate fluorescently labeled secondary antibodies (Life Technologies, CA) and mounted with DAPI containing mounting solution (Vector Laboratories, Burlingame, Calif.). Fluorescence images were acquired using a Zeiss 700 LSM confocal microscope with a 20× objective and analyzed using the Zen software (Carl Zeiss Group, Hartford, Conn.). For transmission electron microscopy (TEM), tissues were fixed, embedded, sectioned, and stained as previously described. Nature Medicine, 2008.) Images were acquired using Zeiss SIGMA HD VP electron microscope.

Statistical Analysis

Data were analyzed using PerkinElmer Columbus and GraphPad Prism software and were compared with using the Student's t-test, where appropriate. P<0.05 was considered statistically significant. Z′-value (Z-prime value), a measure of statistical effect size to determine if the assay response is significant, was calculated as described in literature. (Zhang et al., Journal of Biomolecular Screening, 1999.)

Results

Development of a Podocyte-Based HCS Assay

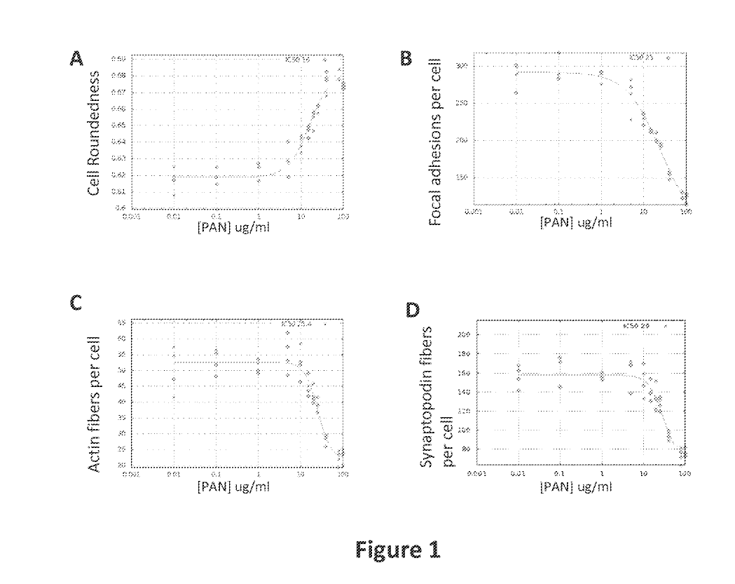

In some embodiments, to quantitatively determine changes in podocyte phenotype with injury, puromycin aminonucleoside (PAN) was used as the damaging agent. PAN-induced nephropathy in rats causes podocyte damage, resulting in podocyte FP effacement and high-grade proteinuria, and is a widely used experimental model for human minimal change disease and/or FSGS. (Pavenstadt et al., Physiol Rev, 2003; Frenk et al. Proc Soc Exp Biol Med, 1955; Caulfield et al., Lab Invest, 1976; Whiteside et al. Am J Pathol, 1993; Pippin et al, Am J Physiol Renal Physiol, 2009.) In vitro, PAN causes podocyte blebbing and rounding, loss of actin stress fibers, reduced focal contacts and adhesion, reactive oxygen species-mediated damage, and eventually, cell death. (Fishman et al., Am J Pathol, 1985; Marshall et al. Kidney Int, 2006.) Podocytes were treated with various doses of PAN and quantified changes in cellular features using our HCS assay system (FIGS. 1 and 2). This comprehensive cellular analysis used images of hundreds of cells per well to quantify various cellular features in an automated and nonbiased fashion. PAN also resulted in increased cell roundness. It shows that a PAN concentration of 16 μg/ml caused half-maximal damage (half-maximal inhibitory concentration), and that 30 mg/ml PAN was sufficient to cause significant podocyte injury. Mizoribine (MZR) is an imidazole nucleoside immunosuppressive agent that has been used in renal transplantation and treatment of steroid-resistant nephrotic syndrome. (Kawasaki, Clin Dev Immunol, 2009.) MZR has also been shown to protect podocytes from PAN injury. (Takeuchi et al. Nephron, Exp Nephrol, 2010.) Treatment of podocytes with MZR showed a dose-dependent protection from PAN-induced injury (30 μg/ml), which was determined from an increase in F-actin fiber count, increased focal adhesions, increased synaptopodin levels, and decreased cell roundness. An MZR concentration of 1.4 μg/ml was sufficient to reduce PAN damage by one half (half-maximal effective concentration) and afforded complete protection of podocytes from PAN injury at 5 μg/ml, which is in good agreement with the reported values in the literature. (Takeuchi et al. Nephron, Exp Nephrol, 2010.) Furthermore, to study if other known podocyte-protective agents would provide similar readouts in our novel assay, we investigated the in vitro effects of glucocorticoid dexamethasone (DEX) on podocytes. Glucocorticoids are widely used to treat a variety of glomerular diseases, and studies have shown that DEX also directly targets and protects podocytes from injury. (Wada et al., J Am Soc Nephrol, 2005; Ransom et al., Kidney Int, 2005; Yamauchi et al. Kidney Int, 2006; Xing et al., Kidney Int, 2006.) Analysis of PAN-injured podocytes treated with DEX showed that DEX provides dose-dependent protection of podocytes from PAN-induced injury (30 μg/ml), which was determined from an increase in F-actin fiber count (FIG. 12), and that DEX as low as 0.1 μM was able to protect cells from PAN damage, which is in excellent agreement with the published literature. (Yamauchi et al. Kidney Int, 2006.) This suggests that this phenotypic HCS assay is quite reliable and not limited to a single agent to quantitatively differentiate damaged podocytes from healthy cells.

To determine the robustness of our optimized assay for use in a screening environment, we evaluated the reproducibility and variability in the assay and analyses by determining the Z′ values across multiple replicates on a plate. (Zhang et al., J Biomol Screen, 1999.) Podocytes in multiple wells were either cultured in the cell culture media or treated with PAN (16 μg/ml) for 48 hours, and the phenotypic parameters for cell roundness and F-actin fibers from each of the wells were quantified. Results show a statistically significant difference (P<0.001) between the PAN and the healthy control cells (FIG. 6). Additionally, the 95% confidence intervals show nice separation for both of the phenotypes. It also shows that the Z′ value for quantified change in a simple cell morphology phenotype (cell roundness) was ≧0.46, suggesting that this simple parameter presented low variability and high compatibility with the HCS environment and high enough robustness for the screening assay. Quantification of F-actin fibers per cell per well provided us with a similar Z′ value (0.44). We also performed a multiparametric analysis using a machine learning algorithm (Linear Classifier in Columbus Analysis Software) to define and phenotypically segment damaged cells from healthy cells within a population (FIG. 13). It also provided highly robust dose-response curves for PAN injury versus healthy controls (FIG. 13B) and an assay Z′ value of 0.69 (FIG. 13C). All of these analyses suggest that a podocyte-based phenotypic assay is amenable to the HCS methodology. On the basis of the data presented above, we chose to use cell roundness as the phenotype for our primary HCS assay with a chemical library to minimize the imaging and image analysis time. Additionally, the podocytes were treated with PAN alone (15-30 μg/ml; negative control) or PAN with 5 μg/ml MZR (positive control) for 48 hours as the optimum conditions for detailed high-content analysis to ensure the capture of a broad range of phenotypic responses.

HCS Assay-Based Identification of Novel Agents that Protect Podocytes from PAN Injury

To identify small molecules that protect podocytes from PAN injury, a chemical library of 2121 pharmacologically active compounds was screened. Podocytes were treated with compounds from the chemical library in the presence of PAN (16 μg/ml) at 37° C. for 48 hours. The assay plates also contained eight positive (PAN and MZR treatment) and eight negative (PAN treatment) control wells. Wells were imaged and analyzed, and the mean cell roundness per cell was calculated for each well. Mean cell roundness was normalized using plate-based positive and negative controls, and a Z score for the change in cell morphology on treatment with each compound (SD away from the plate mean) was calculated. The results from the screen are presented as a graph in FIG. 7A. Wells with nuclei counts. 200 were used in the assay (1985 compounds; average of 576 cells per well) (FIG. 7B), and the rest (136 compounds) were flagged and removed from analyses as potentially highly toxic compounds (frequency plot presented in Supplemental FIG. 4A shows a bell-shaped curve bottoming out at nuclei count <200). The distribution of the Z-score values was bell shaped (frequency plot is shown in FIG. 14B). Primary hits (compounds that protected podocytes from PAN damage) were identified from the assay using a Z-score cutoff of ≦−1.28 corresponding to the 10% population on one side of the bell-shaped curve. As a reference, MZR showed a Z score of −2.31±0.2 in the assay. Our analysis resulted in an initial identification of 34 compounds as primary hits (Table 1, see FIG. 18). Given that our assay is image based, the images from all initial hits were visually inspected manually, which eliminated 10 compounds as nonhits. This resulted in a curated primary hit list of 24 compounds (approximately 1% hit rate). Representative images are shown in FIG. 15. The identified list of primary hit compounds included three Rho kinase inhibitors (Y27632, GSK 429286, and Thiazovivin) and a p38 kinase inhibitor (SB 203580). Inhibition of both Rho kinase and the p38 mitogen-activated protein kinase (MAPK) has previously been shown to protect podocytes from injury in vitro and ameliorate proteinuria in vivo, (Wang et al., Kidney Blood Press Res, 2008; Koshikawa et al., J Am Soc Nephrol, 2005; Pengal et al., Am J Physiol Renal Physiol, 2011) which provides us with excellent internal positive controls and assay validation. Next, to validate the hit compounds identified from our screen, selected primary hits were reanalyzed using independently obtained powder forms of the compounds. All of the chosen hits provided dose-dependent protection of podocytes from PAN injury (FIG. 8), showing the reliability and robustness of the assay.

A Small Molecule Integrin-β1 Agonist Protects Podocytes from Injury

Integrin-α3β1 is highly expressed in podocytes and primarily binds to the GBM-expressed ligand laminin, thereby mediating stable adhesion of podocytes to the GBM. 27 Functional α3β1 is essential for podocyte health and function and maintaining the integrity of podocytes and the glomerular filtration barrier. (Pozzi et al., Dev Biol, 2008; Kanasaki et al., Faul et al., Nat Med, 2008, 2008; Yu et al., N engl J Med, 2013; Tian et al., J Clin Invest; 2014; Krishnamurti et al., J Am Soc Nephrol, 2001; Potla et al., J Clin Invest, 2014.) PAN treatment also reduces expression of α3β1 in podocytes. 32 Damaging agents, protein mutations, or deletions that destabilize active β1 by reducing either its activation or its expression also result in podocyte damage and cause proteinuria in animals and patients. (Yu et al., N Engl J Med, 2013; Tian et al., J Clin Invest; 2014; Krishnamurti et al., J Am Soc Nephrol, 2001; Potla et al., J Clin Invest, 2014.) Therefore, we chose to further validate a top primary hit in our podocyte-based HCS screen (i.e., the small molecule β1-agonist pyrintegrin). (Xu et al., PNAS, 2010.) To test, we investigated the efficacy of pyrintegrin (chemical structure shown in FIG. 9A) in vitro. Pyrintegrin dose dependently protected podocytes from PAN-induced damage, with an apparent half-maximal effective concentration of 0.8 μM (FIG. 8), and showed protection of a variety of cellular markers (FIG. 16). Pyrintegrin also protected podocytes from the loss of actin stress fibers and focal adhesion complexes on PAN-induced injury (FIG. 9B). Interestingly, treatment of healthy cells with pyrintegrin also showed a small but significant increase in actin stress fibers. Given that pyrintegrin affects integrin-β1, (Xu et al., PNAS, 2010.) we quantified the level of β1 in treated cells. We found that PAN reduced β1 levels in podocytes, which were preserved on cotreatment with pyrintegrin (FIG. 16). Furthermore, we also studied the level of active β1 using antibody 9EG7, which specifically recognizes an activation epitope in β1 integrin. (Lenter et al., PNAS, 1993.) Results showed that PAN caused a significant loss of active β1 levels in podocytes but not on treatment with pyrintegrin (FIG. 6C). Treatment of healthy cells with pyrintegrin also showed a significant increase in active β1 levels. Pyrintegrin also showed a similar protection of podocytes from LPS mediated damage (FIG. 17). (Reiser et al., J Clin Invest, 2004.) Finally, to determine whether hits identified from our HCS assay, which is on the basis of murine podocytes, would be applicable to the human system, we tested the efficacy of pyrintegrin in protecting human podocytes from damage. As shown in FIG. 9D, treatment of human podocytes with PAN also produced a significant reduction in the level of active β 1 integrin (as measured by staining with antibody against activation-dependent epitope in human β1 integrin 12G10 (Alachkar et al., N Engl J Med, 2014) that was rescued on treatment with pyrintegrin, suggesting that pyrintegrin is equally effective on human podocytes.

Pyrintegrin Blocks PAN- and LPS-Induced Podocyte Migration

Next, we assessed the efficacy of this small molecule in a functional assay. In vitro, PAN- or LPS-mediated injury promotes directional migration and wound closure by podocytes in a scratch wound-healing assay. (Wei et al., Nat Med, 2008.) Whereas both PAN and LPS treatments significantly increased migration of treated podocytes after 48 hours (FIG. 9E), pyrintegrin cotreatment significantly reduced podocyte motility and wound closure under both conditions. This suggests that pyrintegrin treatment protects podocytes from injury through integrin-β1.

Pyrintegrin Ameliorates Albuminuria In Vivo

Low-dose LPS decreases β1 integrin activation in podocytes and causes podocyte damage and proteinuria in mice. (Yu et al., N Engl J Med, 2013) Here, we used this model to test whether pyrintegrin, by preserving active β1 integrin in podocytes, is able to suppress podocyte FP effacement and the development of proteinuria in live animals. LPS administration into C57BL/6 mice resulted in a strong induction of proteinuria after 24 hours (2.5±0.6 mg albumin per 1 mg creatinine) compared with saline injected animals (0.052±0.022) (FIG. 10A). Ultrastructural analysis showed that it also produced podocyte FP effacement (FIG. 10B). However, coadministration of the β1 agonist pyrintegrin (10 mg/kg) provided a significant protection for these animals from LPS-induced proteinuria (0.668±0.074) (FIG. 10A) and FP effacement (FIG. 10B). Analysis of glomeruli from control animals using confocal microscopy and the β1 activation-specific antibody 9EG7 (and costaining with the podocyte-specific marker synaptopodin (Mundel et al., J Cell Biol, 1997)) showed high expression and localization in the podocytes (FIG. 100). LPS treatment resulted in decreased active β1 integrin and synaptopodin staining in the glomeruli, with very little change in the level of total β1 integrin. In contrast, animals treated with pyrintegrin showed a significant protection of the active β1 levels, similar to its levels in saline-treated control mice. This suggests that the newly identified podocyte-protective compound pyrintegrin is active in vivo and protects animals from proteinuric kidney injury by activation of podocyte β1 integrin. Given that pyrintegrin protected podocytes from PAN-induced damage in vitro, we also tested the efficacy of pyrintegrin in a PAN injury model. Pyrintegrin (10 mg/kg) significantly reduced albuminuria caused by PAN-induced nephropathy in rats (FIG. 7D), further validating the in vitro results, although the level of protection from albuminuria was not as high as that observed in the LPS model system.

Discussion

As shown in FIG. 4A-4P, the initial screen identified the following compounds as agents that prevented PAN induced injury: Pyrintegrin; Kenpoullon; Kinetin; Apigenin; Agelasine; Berberine; Derusnin; Ethaverine; Diosmetin; Doxazocin; Thioguanidine; P38 inhibitor (SB239063); FR 180204; Mundeserone; Y27632; Alk5 Inhibitor2; and GSK429286.

Potential molecular targets of identified hits include: Rho-kinase (ROCK) inhibitors (Y27632; GSK429286); P38 inhibitors (SB 239063); ERK1/2 inhibitors (FR 180204); KLF4 mimic (alternatively GSK3b inhibitor, CDK5 inhibitor); (Kenpaullone; Alsterpaullone); Intergin beta1 agonist (Pyrintegrin); PARK1 agonist (Kinetin; Kinetin riboside).

Podocyte drug discovery is hampered, because the techniques for rapid identification of podocyte-targeting agents have been lacking. Here, we describe the first podocyte cell-based phenotypic assay for high throughput HCS of agents for the identification of podocyte-protective therapeutics. This assay uses a confocal microscopy-based HCS system to image and measure phenotypic changes in cultured podocytes, such as cell morphology, F-actin cytoskeleton, and focal adhesions, that have been used in the past to define healthy podocytes to provide a robust, unbiased, and quantitative readout that has established relevance to podocyte function in vivo. Reiser et al., Annu Rev Med, 2013.) A few attempts in the past at designing HTS assays relevant to podocytes used stably transfected cells with a nephrin promoter-based reporter for the identification of a few small molecules that modulate the nephrin expression. (Yamauchi et al., Kidney Int, 2006.) However, cell based phenotypic assays have significant advantages over more target-based approaches and have been more successful in generating novel Food and Drug Administration-approved therapeutics for various indications. (Swinney et al., Nat Rev Drug Discov, 2011; Lee et al., J Biomol Screen, 2013.) Using PAN-induced injury as a model system, we show that the assay provides quantitative assessment of podocyte damage and has lowvariability and high robustness for use in an HTS environment. Furthermore, on screening with a >2100 compound chemical library, we identified 24 small molecules that protected podocytes from PAN injury. Inhibition of signaling proteins Rho-associated protein kinase (ROCK) and p38 MAPK using small molecule antagonists has previously been shown to protect against podocyte injury in vitro and proteinuria in vivo. (Wang et al., Kidney Blood Press Res, 2008; Koshikawa et al., J Am Soc Nephrol, 2005; Hidaka et al., Am J Pathol, 2008; Kanda et al., Kidney Int, 2003; Nishikimi et al., J Pharmacol Sci, 2006; Sakurai et al., Nephrol Dial Transplant, 2009; Shibata et al., J Am Soc Nephrol, 2006.) Among the primary hits, we found compounds that inhibit ROCK (Y27632, GSK 429286, and Thiazovivin) or p38 MAPK (SB 203580), which provided us with excellent validation for the screening assay. Furthermore, the assays showed Z′ values of 0.44 and 0.46, respectively, in two independent primary assay readouts and Z′>0.65 for a multiparametric classifier assay readout. Although Z′ values >0.5 are generally required for use of a biochemical assay in an HTS campaign, HCS assays are considered ready for HTS with lower Z′ values because of the variability and complexities of the multiple image-based readouts. (Quintavalle et al., Sci Signal, 2011; Singh et al., J Biomol Screen, 2014.) Additionally, although the primary screen presented in this report used cells of the murine origin, which show a more homogenous phenotype for better quantification in a phenotypic assay, the assay methodology described here is applicable to human podocytes as well (FIG. 9D). The β1 integrin α3β1 is highly relevant for podocytes and the integrity of the filtration barrier in vivo. (Pozzi et al., J Am Soc Nephrol, 2013; Pozzi et al, Dev Biol, 2008; Kanasaki et al., Dev Biol, 2008.) Global or podocyte-specific deletion of either subunit leads to proteinuria and early death in animals. (Pozzi et al, Dev Biol, 2008; Kanasaki et al., Dev Biol, 2008; Sachs et al. J Clin Invest, 2012.) Additionally, we and others have recently shown that podocyte injury leads to reduced levels of active β1 in mice and patients, (Yu et al. N Engl J Med, 2013; Potla et al., J Clin Invest, 2014) which suggests that preventing loss of active β1-integrin levels in podocytes might be protective. Using novel small molecule agonists of β2 integrin CD11b/CD18, we have previously shown that such pharmacologic activation of integrins in vivo is a new, therapeutically relevant mechanism for targeting this family of adhesion receptors. (Maiguel et al., Sci Signal, 2011.) Identified by our screen, pyrintegrin provided dose-dependent protection to cells from PAN injury in vivo and in vitro. Most importantly, it protected mice from LPS-induced proteinuria and podocyte FP effacement. Pyrintegrin administration for 14 days also significantly reduced peak proteinuria in PAN-induced nephropathy, albeit with smaller effects than observed in the LPS model. A reason for this effect could be the low solubility of pyrintegrin in the aqueous media, suggesting that future optimization of pyrintegrin might be warranted for chronic delivery.

In addition to pyrintegrin, we identified inhibitor totaling 24 primary hits, including apigenin (a flavone that may induce autophagy), agelasine (inhibitor of Na/K-ATPase), antimycin A (antifungal; depletes ATP and has been shown to induce F-actin polymerization (Atkinson et al., J Biol Chem, 2004), berberine (antifungal isoquinoline alkaloid with wide activities), doxazocin (a quinazoline antihypertensive α1-selective α-blocker), and thioguanine (antiproliferative and cytotoxic). We also identified two synthetic progestins altrenogest and norgestimate. Podocytes express hormone receptors, including for progesterone and estrogen, and 17β-estradiol has been shown to stabilize actin cytoskeleton and protect podocytes against oxidant-induced injury. (Catanuto et al., Kidney Int, 2009; Doublier et al., Kidney Int, 2011; Catanuto et al., Endocrinology, 2012.) The diversity of the identified hits signifies the possibility of identifying a number of relevant signaling pathways through the presented phenotypic assay that may be targeted for protecting podocytes from injury.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

### REFERENCES

1. S. Takeuchi et al., The immunosuppressive drug mizoribine directly prevents podocyte injury in puromycin aminonucleoside nephrosis. Nephron. Experimental nephrology 116, e3 (2010).
2. S. Y. Gao et al., Rho-ROCK signal pathway regulates microtubule-based process formation of cultured podocytes—inhibition of ROCK promoted process elongation. Nephron. Experimental nephrology 97, e49 (2004).
3. M. Koshikawa et al., Role of p38 mitogen-activated protein kinase activation in podocyte injury and proteinuria in experimental nephrotic syndrome. Journal of the American Society of Nephrology: JASN 16, 2690 (September, 2005).
4. D. W. Zaharevitz et al., Discovery and initial characterization of the paullones, a novel class of small-molecule inhibitors of cyclin-dependent kinases. Cancer research 59, 2566 (Jun. 1, 1999).
5. C. A. Lyssiotis et al., Reprogramming of murine fibroblasts to induced pluripotent stem cells with chemical complementation of Klf4. Proceedings of the National Academy of Sciences of the United States of America 106, 8912 (Jun. 2, 2009).
6. M. Yamada et al., Inhibition of protein kinase CK2 prevents the progression of glomerulonephritis. Proceedings of the National Academy of Sciences of the United States of America 102, 7736 (May 24, 2005).
7. N. J. Linford, Y. Yang, D. G. Cook, D. M. Dorsa, Neuronal apoptosis resulting from high doses of the isoflavone genistein: role for calcium and p42/44 mitogen-activated protein kinase. The Journal of pharmacology and experimental therapeutics 299, 67 (October, 2001).
8. Y. Xu et al., Revealing a core signaling regulatory mechanism for pluripotent stem cell survival and self-renewal by small molecules. Proceedings of the National Academy of Sciences of the United States of America 107, 8129 (May 4, 2010).
9. J. A. Kreidberg et al., Alpha 3 beta 1 integrin has a crucial role in kidney and lung organogenesis. Development 122, 3537 (November, 1996).
10. A. Pozzi et al., Beta1 integrin expression by podocytes is required to maintain glomerular structural integrity. Developmental biology 316, 288 (Apr. 15, 2008).
11. Pavenstädt H, Kriz W, Kretzler M: Cell biology of the glomerular podocyte. Physiol Rev 83: 253-307, 2003
12. Faul C, Asanuma K, Yanagida-Asanuma E, Kim K, Mundel P: Actin up: Regulation of podocyte structure and function by components of the actin cytoskeleton. Trends Cell Biol 17: 428-437, 2007
13. Huber T B, Benzing T: The slit diaphragm: A signaling platform to regulate podocyte function. Curr Opin Nephrol Hypertens 14: 211-216, 2005
14. Reiser J, Sever S: Podocyte biology and pathogenesis of kidney disease. Annu Rev Med 64: 357-366, 2013
15. Wiggins R C: The spectrum of podocytopathies: A unifying view of glomerular diseases. Kidney Int 71: 1205-1214, 2007
16. Mathieson P W: The podocyte as a target for therapies—new and old. Nat Rev Nephrol 8: 52-56, 2012
17. Reiser J, Gupta V, Kistler A D: Toward the development of podocyte-specific drugs. Kidney Int 77: 662-668, 2010
18. Faul C, Donnelly M, Merscher-Gomez S, Chang Y H, Franz S, Delfgaauw J, Chang J M, Choi H Y, Campbell K N, Kim K, Reiser J, Mundel P: The actin cytoskeleton of kidney podocytes is a direct target of the anti-proteinuric effect of cyclosporine A. Nat Med 14: 931-938, 2008
19. Mundel P, Reiser J, Zuñiga Mejía Borja A, Pavenstädt H, Davidson G R, Kriz W, Zeller R: Rearrangements of the cytoskeleton and cell contacts induce process formation during differentiation of conditionally immortalized mouse podocyte cell lines. Exp Cell Res 236: 248-258, 1997
20. Saleem M A, O'Hare M J, Reiser J, Coward R J, Inward C D, Farren T, Xing C Y, Ni L, Mathieson P W, Mundel P: A conditionally immortalized human podocyte cell line demonstrating nephrin and podocin expression. J Am Soc Nephrol 13: 630-638, 2002
21. Frenk S, Antonowicz I, Craig J M, Metcoff J: Experimental nephrotic syndrome induced in rats by aminonucleoside; renal lesions and body electrolyte composition. Proc Soc Exp Biol Med 89: 424-427, 1955
22. Caulfield J P, Reid J J, Farquhar M G: Alterations of the glomerular epithelium in acute aminonucleoside nephrosis. Evidence for formation of occluding junctions and epithelial cell detachment. Lab Invest 34: 43-59, 1976
23. Whiteside C I, Cameron R, Munk S, Levy J: Podocytic cytoskeletal disaggregation and basement-membrane detachment in puromycin aminonucleoside nephrosis. Am J Pathol 142: 1641-1653, 1993
24. Pippin J W, Brinkkoetter P T, Cormack-Aboud F C, Durvasula R V, Hauser P V, Kowalewska J, Krofft R D, Logar C M, Marshall C B, Ohse T, Shankland S J: Inducible rodent models of acquired podocyte diseases. Am J Physiol Renal Physiol 296: F213-F229, 2009
25. Fishman J A, Kamovsky M J: Effects of the aminonucleoside of puromycin on glomerular epithelial cells in vitro. Am J Pathol 118: 398-407, 1985
26. Marshall C B, Pippin J W, Krofft R D, Shankland S J: Puromycin aminonucleoside induces oxidant-dependent DNA damage in podocytes in vitro and in vivo. Kidney Int 70: 1962-1973, 2006
27. Kawasaki Y: Mizoribine: A new approach in the treatment of renal disease. Clin Dev Immunol 2009: 681482, 2009
28. Takeuchi S, Hiromura K, Tomioka M, Takahashi S, Sakairi T, Maeshima A, Kaneko Y, Kuroiwa T, Nojima Y: The immunosuppressive drug mizoribine directly prevents podocyte injury in puromycin aminonucleoside nephrosis. Nephron, Exp Nephrol 116: e3-e10, 2010
29. Wada T, Pippin J W, Marshall C B, Griffin S V, Shankland S J: Dexamethasone prevents podocyte apoptosis induced by puromycin aminonucleoside: Role of p53 and Bcl-2-related family proteins. J Am Soc Nephrol 16:2615-2625, 2005
30. Ransom R F, Lam N G, Hallett M A, Atkinson S J, Smoyer W E: Glucocorticoids protect and enhance recovery of cultured murine podocytes via actin filament stabilization. Kidney Int 68: 2473-2483, 2005
31. Yamauchi K, Takano Y, Kasai A, Hayakawa K, Hiramatsu N, Enomoto N, Yao J, Kitamura M: Screening and identification of substances that regulate nephrin gene expression using engineered reporter podocytes. Kidney Int 70: 892-900, 2006
32. Xing C Y, Saleem M A, Coward R J, Ni L, Witherden I R, Mathieson P W: Direct effects of dexamethasone on human podocytes. Kidney Int 70: 1038-1045, 2006
33. Zhang J H, Chung T D, Oldenburg K R: A simple statistical parameter for use in evaluation and validation of high throughput screening assays. J Biomol Screen 4: 67-73, 1999
34. Wang L, Ellis M J, Fields T A, Howell D N, Spumey R F: Beneficial effects of the Rho kinase inhibitor Y27632 in murine puromycin aminonucleoside nephrosis. Kidney Blood Press Res 31: 111-121, 2008
35. Koshikawa M, Mukoyama M, Mori K, Suganami T, Sawai K, Yoshioka T, Nagae T, Yokoi H, Kawachi H, Shimizu F, Sugawara A, Nakao K: Role of p38 mitogen-activated protein kinase activation in podocyte injury and proteinuria in experimental nephrotic syndrome. J Am Soc Nephrol 16: 2690-2701, 2005
36. Pengal R, Guess A J, Agrawal S, Manley J, Ransom R F, Mourey R J, Benndorf R, Smoyer W E: Inhibition of the protein kinase MK-2 protects podocytes from nephrotic syndrome-related injury. Am J Physiol Renal Physiol 301: F509-F519, 2011
37. Pozzi A, Zent R: Integrins in kidney disease. J Am Soc Nephrol 24: 1034-1039, 2013
38. Kanasaki K, Kanda Y, Palmsten K, Tanjore H, Lee S B, Lebleu V S, Gattone V H Jr., Kalluri R: Integrin beta1-mediated matrix assembly and signaling are critical for the normal development and function of the kidney glomerulus. Dev Biol 313: 584-593, 2008
39. Yu C C, Fomoni A, Weins A, Hakroush S, Maiguel D, Sageshima J, Chen L, Ciancio G, Faridi M H, Behr D, Campbell K N, Chang J M, Chen H C, Oh J, Faul C, Amaout M A, Fiorina P, Gupta V, Greka A, Burke G W 3rd, Mundel P: Abatacept in B7-1-positive proteinuric kidney disease. N Engl J Med 369, 2416-2423, 2013
40. Tian X, Kim J J, Monkley S M, Gotoh N, Nandez R, Soda K, Inoue K, Balkin D M, Hassan H, Son S H, Lee Y, Moeckel G, Calderwood D A, Holzman L B, Critchley D R, Zent R, Reiser J, Ishibe S: Podocyte-associated talin1 is critical for glomerular filtration barrier maintenance. J Clin Invest 124, 1098-1113, 2014
41. Krishnamurti U, Zhou B, Fan W W, Tsilibary E, Wayner E, Kim Y, Kashtan C E, Michael A: Puromycin aminonucleoside suppresses integrin expression in cultured glomerular epithelial cells. J Am Soc Nephrol 12: 758-766, 2001
42. Potla U, Ni J, Vadaparampil J, Yang G, Leventhal J S, Campbell K N, Chuang P V, Morozov A, He J C, D'Agati V D, Klotman P E, Kaufman L: Podocyte-specific RAP1GAP expression contributes to focal segmental glomerulosclerosis-associated glomerular injury. J Clin Invest 124: 1757-1769, 2014
43. Lenter M, Uhlig H, Hamann A, Jeno P, Imhof B, Vestweber D: A monoclonal antibody against an activation epitope on mouse integrin chain beta 1 blocks adhesion of lymphocytes to the endothelial integrin alpha 6 beta 1. Proc Natl Acad Sci USA 90: 9051-9055, 1993
44. Reiser J, von Gersdorff G, Loos M, Oh J, Asanuma K, Giardino L, Rastaldi M P, Calvaresi N, Watanabe H, Schwarz K, Faul C, Kretzler M, Davidson A, Sugimoto H, Kalluri R, Sharpe A H, Kreidberg J A, Mundel P: Induction of B7-1 in podocytes is associated with nephrotic syndrome. J Clin Invest 113: 1390-1397, 2004
45. Alachkar N, Carter-Monroe N, Reiser J: Abatacept in B7-1-positive proteinuric kidney disease. N Engl J Med 370: 1263-1264, 2014
46. Wei C, Möller C C, Altintas M M, Li J, Schwarz K, Zacchigna S, Xie L, Henger A, Schmid H, Rastaldi M P, Cowan P, Kretzler M, Parrilla R, Bendayan M, Gupta V, Nikolic B, Kalluri R, Carmeliet P, Mundel P, Reiser J: Modification of kidney barrier function by the urokinase receptor. Nat Med 14: 55-63, 2008
47. Mundel P, Heid H W, Mundel T M, Krüger M, Reiser J, Kriz W: Synaptopodin: An actin-associated protein in telencephalic dendrites and renal podocytes. J Cell Biol 139: 193-204, 1997
48. Swinney D C, Anthony J: How were new medicines discovered? Nat Rev Drug Discov 10: 507-519, 2011
49. Lee J A, Berg E L: Neoclassic drug discovery: The case for lead generation using phenotypic and functional approaches. J Biomol Screen 18: 1143-1155, 2013
50. Hidaka T, Suzuki Y, Yamashita M, Shibata T, Tanaka Y, Horikoshi S, Tomino Y: Amelioration of crescentic glomerulonephritis by RhoA kinase inhibitor, Fasudil, through podocyte protection and prevention of leukocyte migration. Am J Pathol 172: 603-614, 2008
51. Kanda T, Wakino S, Hayashi K, Homma K, Ozawa Y, Saruta T: Effect of fasudil on Rho-kinase and nephropathy in subtotally nephrectomized spontaneously hypertensive rats. Kidney Int 64: 2009-2019, 2003
52. Nishikimi T, Matsuoka H: Molecular mechanisms and therapeutic strategies of chronic renal injury: Renoprotecttve effect of rho-kinase inhibitor in hypertensive glomerulosclerosis. J Pharmacol Sci 100: 22-28, 2006
53. Sakurai N, Kuroiwa T, Ikeuchi H, Hiramatsu N, Takeuchi S, Tomioka M, Shigehara T, Maeshima A, Kaneko Y, Hiromura K, Kopp J B, Nojima Y: Fluvastatin prevents podocyte injury in a murine model of HIV-associated nephropathy. Nephrol Dial Transplant 24: 2378-2383, 2009
54. Shibata S, Nagase M, Fujita T: Fluvastatin ameliorates podocyte injury in proteinuric rats via modulation of excessive Rho signaling. J Am Soc Nephrol 17: 754-764, 2006
55. Quintavelle M, Elia L, Price J H, Heynen-Genel S, Courtneidge S A: A cell-based high-content screening assay reveals activators and inhibitors of cancer cell invasion. Sci Signal 4: ra49, 2011
56. Singh S, Carpenter A E, Genovesio A: Increasing the content of high-content screening: An overview. J Biomol Screen 19: 640-650, 2014
57. Sachs N, Claessen N, Aten J, Kreft M, Teske G J, Koeman A, Zuurbier C J, Janssen H, Sonnenberg A: Blood pressure influences end-stage renal disease of Cd151 knockout mice. J Clin Invest 122: 348-358, 2012
58. Maiguel D, Faridi M H, Wei C, Kuwano Y, Balla K M, Hemandez D, Barth C J, Lugo G, Donnelly M, Neyer A, Moita L F, Schürer S, Traver D, Ruiz P, Vazquez-Padron R I, Ley K, Reiser J, Gupta V: Small molecule-mediated activation of the integrin CD11b/CD18 reduces inflammatory disease. Sci Signal 4: ra57, 2011
59. Atkinson S J, Hosford M A, Molitoris B A: Mechanism of actin polymerization in cellular ATP depletion. J Biol Chem 279: 5194-5199, 2004
60. Catanuto P, Doublier S, Lupia E, Foment A, Berho M, Karl M, Striker G E, Xia X, Elliot S: 17 beta-estradiol and tamoxifen upregulate estrogen receptor beta expression and control podocyte signaling pathways in a model of type 2 diabetes. Kidney Int 75: 1194-1201, 2009
61. Doublier S, Lupia E, Catanuto P, Periera-Simon S, Xia X, Korach K, Berho M, Elliot S J, Karl M: Testosterone and 17β-estradiol have opposite effects on podocyte apoptosis that precedes glomerulosclerosis in female estrogen receptor knockout mice. Kidney Int 79: 404-413, 2011
62. Catanuto P, Fornoni A, Pereira-Simon S, Wu F, Burnstein K L, Xia X, Conti F, Lenzi A, Elliot S: In vivo 17β-estradiol treatment contributes to podocyte actin stabilization in female db/db mice. Endocrinology 153: 5888-5895. 2012

1. A method of screening an agent for the treatment of kidney disease, the method comprising:
culturing podocytes in vitro;
adding a test agent to the podocyte culture and culturing the podocyte culture with the test agent;
observing the podocyte culture in contact with the test agent using a readout system relative to a control podocyte culture to measure one or more markers of podocyte protection; and
identifying the test agent that protects the podocyte culture from injury.

2. The method according to claim 1, wherein the readout system comprises a microcopy based readout.

3. The method according to claim 1 or 2, wherein the one or more markers is selected from the group consisting of cell morphology, cell roundedness, F-actin intensity, number of focal adhesions, intensity or size of focal adhesions, length of actin fibers, size of actin fibers, number of actin fibers per cell, number of synaptopodin fibers per cell and β1 integrin levels.

4. The method according to claim 1, comprising culturing the podocytes in a multiwell plate.

5. The method according to claim 4, comprising differentiating the podocytes prior to culturing the podocytes in the multiwell plate.

6. The method according to claim 1, wherein the control culture comprises a negative control podocyte culture comprising puromycin anminonucleoside (PAN) treatment.

7. The method according to claim 1, wherein the control culture comprises a positive control podocyte culture comprising PAN and mizoribine (MZR) treatment.

8. The method according to claim 1, wherein the control culture comprises a positive control podocyte culture comprising PAN and dexamethasone (DEX) treatment.

9. The method according to claim 1, comprising culturing the podocyte culture with the test agent and PAN in the podocyte culture.

10. The method according to claim 1, comprising screening a library of test agents.

11. (canceled)

12. A method of identifying a kidney disease causing agent in a patient's sample, the method comprising:
isolating a biological sample from the patient;
adding the biological sample to a podocyte culture in vitro and culturing the podocytes with the biological sample;
measuring one or more cell markers relative to a control; and
identifying a disease causing agent in the patient's biological sample when the percent damaged cells in the podocyte culture with the biological sample is greater than the control.

13. The method of claim 12, comprising measuring one or more of the following markers selected from the group consisting of cell morphology, cell roundedness, F-actin intensity, number of focal adhesions, intensity or size of focal adhesions, length of actin fibers, size of actin fibers, number of actin fibers per cell, number of synaptopodin fibers per cell and β1 integrin levels.

14. The method according to claim 11, comprising differentiating the podocytes prior to culturing the podocytes in a multiwell plate.

15. The method according to claim 11, comprising measuring the one or more biomarkers using confocal microscopy.

16. A method of treating kidney disease or protecting podocytes from injury, the method comprising administering to a patient in need thereof a therapeutically effective amount of at least one agent from the group consisting of Pyrintegrin; Kenpoullon; Kinetin; Apigenin; Agelasine; Berberine; Derusnin; Ethaverine; Diosmetin; Doxazocin; Thioguanidine; P38 inhibitor (SB239063); FR 180204; Mundeserone; Y27632; Alk5 Inhibitor2; Alsterpaullone and GSK429286.

17. (canceled)

18. The method according to claim 16, wherein the at least one agent comprises Pyrintegrin
